(19) United States
(12) Patent Application Publication
(10) Pub. No.: US 2026/0016474 A1
(43) Pub. Date: Jan. 15, 2026

(54) METHODS AND PRODUCTS FOR DETERMINING RESPONSIVENESS TO ANTI-PD1 IMMUNE CHECKPOINT INHIBITOR IMMUNOTHERAPY

(71) Applicants: University of South Australia, Adelaide (AU); Central Adelaide Local Health Network Inc, Adelaide (AU)

(72) Inventors: Lisa Michelle Ebert, Adelaide (AU); Michael Paul Brown, Adelaide (AU); Nga Thi Hong Truong, Adelaide (AU)

(73) Assignees: University of South Australia, Adelaide (AU); Central Adelaide Local Health Network Inc, Adelaide (AU)

(21) Appl. No.: 18/994,428

(22) PCT Filed: Jul. 14, 2023
(86) PCT No.: PCT/AU2023/050654

§ 371 Date: Jan. 14, 2025

(30) Foreign Application Priority Data
Jul. 14, 2022 (AU) 2022901967

(51) Int. Cl. G01N 33/574; G01N 15/1429

(52) U.S. Cl. CPC G01N 33/57484; G01N 15/1429; G01N 2333/70521; G01N 2333/70596

(57) ABSTRACT

The present invention provides methods, systems, and kits for determining responsiveness of a subject with or susceptible to cancer to anti-PD1 immune checkpoint inhibitor immunotherapy (ICII) including determining the level of one or more of markers CD15 in CD8+Ki67/CD71+ or CD8/CD4−Ki67/CD71+ T cells, CTLA-4 in CD4+Ki67/CD71+ T cells, FoxP3 in CD4+Ki67/CD71+ T cells, Ki67 or CD71 in CD8+ T cells, wherein an increase in CD15, CTLA-4, FoxP3, or a decrease in Ki67 or CD71 prior to immunotherapy is indicative that the subject is responsive to immunotherapy, particularly PD1 ICII.

This application claims priority from Australian provisional patent application number 2022901967 filed on 14 Jul. 2022, the entire disclosure of which is incorporated herein by this reference.

### FIELD

The present disclosure relates to methods and products for determining the responsiveness of a subject suffering from, or susceptible to, a cancer to anti-PD1 immune checkpoint inhibitor immunotherapy.

### BACKGROUND

Immune Checkpoint Inhibitor (ICI) therapy is emerging as an important new approach to cancer treatment. ICI drugs are monoclonal antibodies that block the function of certain molecules, known as immune checkpoints, which normally act to suppress T cell function. By deactivating these checkpoints, anti-tumour immunity is reinvigorated and cancer cells become subject to intense immune-mediated attack, resulting in tumour shrinkage.

Immune checkpoint inhibitor therapy is now used routinely in many countries for the treatment of melanoma, lung cancer and many other types of cancer. In Australia, immune checkpoints currently targeted clinically in cancer patients are PD1 (inhibited by pembrolizumab and nivolumab) and its ligand PD-L1 (inhibited by avelumab and atezolizumab), and CTLA4 (inhibited by ipilimumab), although antibodies targeting additional checkpoints are under pre-clinical and clinical development. These drugs have recently been recently approved for the treatment of patients with melanoma, non-small cell lung cancer (NSCLC), Merkel cell carcinoma, renal cell carcinoma, head and neck cancer and Hodgkin lymphoma.

Following treatment with ICI drugs, some patients experience complete and long-lasting tumour regression. However, other patients respond only poorly or not at all. Despite being used routinely for the treatment of many types of cancer, ICI therapy produces substantial and durable therapeutic effects in only a minor proportion of treated patients, while the majority derive no or only transient benefit.

In addition, considering the important role that these immune checkpoints play in negative regulation of the immune system, it is not surprising that this approach can also be associated with the development of autoimmune toxicities, which can be severe in some patients.

Despite intense investigation over the last few years, no practical approach has yet been developed to accurately predict response to therapy. Furthermore, because ICI drugs frequently lead to autoimmune toxicities, which can be severe, many cancer patients receive a very expensive and potentially toxic therapy that will fail to treat their cancer.

Accordingly, there is a need for the development of methods to predict ICI responsiveness, in particular to assist with the selection of personalised treatment decisions for patients who may or may not respond to the therapy.

### SUMMARY

The present disclosure relates to methods and products for determining the responsiveness of a subject suffering from, or susceptible to, a cancer to immune checkpoint inhibitor immunotherapy.

Certain embodiments of the present disclosure provide a method of determining responsiveness of a subject suffering from, or susceptible to, a cancer to anti-PD1 immune checkpoint inhibitor immunotherapy, the method comprising assessing the vascular and/or tumour associated level in the subject of one or more of CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells, wherein an increased level of the CD15s marker in the Ki67+CD8+ T cells prior to the immunotherapy, an increased level of the CTLA-4 marker in said T cells prior to and/or after the immunotherapy, an increased level of the FoxP3 marker in said T cells prior to immunotherapy, an increased level of the CD15s marker in Ki67+CD4− CD8− T cells prior to immunotherapy, and a decreased level of the Ki67+ marker in said T cells prior to immunotherapy, is indicative that the subject is responsive to the immunotherapy.

Certain embodiments of the present disclosure provide a method of determining responsiveness of a subject suffering from, or susceptible to, a cancer to anti-PD1 immune checkpoint inhibitor immunotherapy, the method comprising assessing the level in the blood in the subject of one or more of CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells, wherein an increased level of the CD15s marker in the Ki67+CD8+ T cells prior to the immunotherapy, an increased level of the CTLA-4 marker in said T cells prior to and/or after the immunotherapy, an increased level of the FoxP3 marker in said T cells prior to immunotherapy, an increased level of the CD15s marker in Ki67+CD4− CD8− T cells prior to immunotherapy, and a decreased level of the Ki67+ in said T cells marker prior to immunotherapy, is indicative that the subject is responsive to the immunotherapy.

Certain embodiments of the present disclosure provide a method of determining responsiveness of a subject suffering from, or susceptible to, a cancer to anti-PD1 immune checkpoint inhibitor immunotherapy, the method comprising assessing the level in cells in a tumour in the subject of one or more of CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells, wherein an increased level of the CD15s marker in the Ki67+CD8+ T cells prior to the immunotherapy, an increased level of the CTLA-4 marker in said T cells prior to and/or after the immunotherapy, an increased level of the FoxP3 marker in said T cells prior to immunotherapy, an increased level of the CD15s marker in Ki67+CD4− CD8− T cells prior to immunotherapy, and a decreased level of the Ki67+ marker in said T cells prior to immunotherapy, is indicative that the subject is responsive to the immunotherapy.

Certain embodiments of the present disclosure provide a method of determining responsiveness of a subject suffering from, or susceptible to, a cancer to anti-PD1 immune checkpoint inhibitor immunotherapy, the method comprising assessing the level in tumour infiltrating lymphocytes in the subject of one or more of CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells, wherein an increased level of the CD15s marker in the Ki67+CD8+ T cells prior to the immunotherapy, an increased level of the CTLA-4 marker in said T cells prior to and/or after the immunotherapy, an increased level of the FoxP3 marker in said T cells prior to immunotherapy, an increased level of the CD15s marker in Ki67+CD4− CD8− T cells prior to immunotherapy, and a decreased level of the Ki67+ marker in said T cells prior to immunotherapy, is indicative that the subject is responsive to the immunotherapy.

Certain embodiments of the present disclosure provide a method of treating a subject suffering from, or susceptible to, a cancer with an anti-PD1 immune checkpoint inhibitor immunotherapy, the method comprising identifying a subject responsive to the immune checkpoint inhibitor immunotherapy using a method as described herein and treating the subject so identified with the immune checkpoint inhibitor immunotherapy.

Certain embodiments of the present disclosure provide a method of treating a subject suffering from, or susceptible to, a cancer with an anti-PD1 immune checkpoint inhibitor immunotherapy, the method comprising:
determining the vascular and/or tumour associated level of one or more of one or more of CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells, wherein an increased level of the CD15s marker in the Ki67+CD8+ T cells prior to the immunotherapy, an increased level of the CTLA-4 marker in said T cells prior to and/or after the immunotherapy, an increased level of the FoxP3 marker in said T cells prior to immunotherapy, an increased level of the CD15s marker in Ki67+CD4− CD8− T cells prior to immunotherapy, and a decreased level of the Ki67+ marker in said T cells prior to immunotherapy, is indicative that the subject is responsive to the immunotherapy; and
treating the subject so determined to be responsive with the immunotherapy.

Certain embodiments of the present disclosure provide a kit for use in assessing responsiveness of a subject suffering from, or susceptible to, a cancer to anti-PD1 immune checkpoint inhibitor immunotherapy; the kit comprising one or more of an immunological reagent for detecting CD15s marker, an immunological reagent for detecting CTLA-4 marker, an immunological reagent for detecting FoxP3 marker, and an immunological reagent for detecting Ki67 marker.

Certain embodiments of the present disclosure provide use of one or more of CD15s, CTLA-4, FoxP3 and Ki67 as a marker to assess the responsiveness of a subject suffering from, or susceptible to, a cancer to anti-PD1 immune checkpoint inhibitor immunotherapy.

Certain embodiments of the present disclosure provide a method of assessing responsiveness of a subject suffering from, or susceptible to, a cancer to anti-PD1 immune checkpoint inhibitor immunotherapy, the method comprising:
receiving data representative of the vascular and/or tumour associated level of one or more of CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells; and
processing the data to determine the responsiveness of the subject to the immunotherapy.

Certain embodiments of the present disclosure provide software for use with a computer comprising a processor and memory for storing the software, the software comprising a series of instructions executable by the processor to carry out a method as described herein.

Certain embodiments of the present disclosure provide a system for assessing responsiveness of a subject suffering from, or susceptible to, a cancer to anti-PD1 immune checkpoint inhibitor immunotherapy, the system comprising:
a flow cytometer;
a processor;
a memory; and
software resident in the memory accessible to the processor, the software comprising a series of instructions executable by the processor to carry out method a method as described herein.

Certain embodiments of the present disclosure provide a system for assessing responsiveness of a subject suffering from, or susceptible to, a cancer to anti-PD1 immune checkpoint inhibitor immunotherapy, the system comprising:
a means for determining the level of one or more of CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells, in a blood and/or tumour associated sample from a subject;
a processor;
a memory; and
software resident in the memory accessible to the processor, the software comprising a series of instructions executable by the processor to process data representative of the vascular and/or tumour associated level of the one or more of the markers, and thereby provide a measure of the responsiveness of the subject to the immunotherapy.

Certain embodiments of the present disclosure provide a system for assessing responsiveness of a subject suffering from, or susceptible to, a cancer to anti-PD1 immune checkpoint inhibitor immunotherapy, the system comprising
a flow cytometer;
a processor;
a memory; and
software resident in the memory accessible to the processor, the software comprising a series of instructions executable by the processor to process data received from the flow cytometer representative of the vascular and/or tumour associated level of the CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells, and thereby provide a measure of the responsiveness of the subject to the immunotherapy.

In certain embodiments of the methods and systems of the invention, the Ki67+CD8+ T cells comprise Ki67+CD8+CD3+ T cells.

In certain embodiments of the methods and systems of the invention, the Ki67+CD8+ T cells comprise Ki67+CD8+CD3+CD28+ T cells

In certain embodiments of the methods and systems of the invention, the Ki67+CD4+ T cells comprise Ki67+CD4+CD8− T cells.

In certain embodiments of the methods and systems of the invention, the Ki67+CD4+ T cells comprise Ki67+CD4+CD3+ T cells.

In certain embodiments, the method comprises determining the level of one or more of the markers expressed by cells by flow cytometry.

In certain embodiments the method comprises use of one or more of FH6, HECA-452, CHO131, and CSLEX monoclonal antibodies to determine the level of the CD15s marker in Ki67+CD8+ T cells.

In certain embodiments of the methods and systems of the invention, a level of the CD15s marker in Ki67+CD8+ T cells of 2% or greater, preferrably 3.5% or greater, determined using the FH6 monoclonal antibody (mAb) in a sample from the subject prior to immunotherapy, is indicative that the subject is responsive to the immunotherapy.

In certain embodiments of the methods and systems of the invention, a level of the CD15s marker in Ki67+CD8+ T cells of 10% or greater, determined using the HECA-452 mAb in a sample from the subject prior to immunotherapy, is indicative that the subject is responsive to the immunotherapy.

In certain embodiments of the methods and systems of the invention, a level of the CD15s marker in Ki67+CD8+ T cells of 18% or greater, determined using the CHO131 mAb in a sample from the subject prior to immunotherapy, is indicative that the subject is responsive to the immunotherapy.

In certain embodiments the marker CD71 can be used in place of Ki67 as an indicator of cell proliferation. In other embodiment CD71 can be used in combination with Ki67 to detect cells which are both CD71+ and Ki67+. While explicit disclosure is provided for Ki67+ and CD71+ cell separately, each of the disclosures of CD71+ cells should be taken to include dual positive cells (i.e., Ki67+ and CD71+ cells)

In certain embodiments of the methods and systems of the invention, a level of the CD15s marker in Ki67+CD8+ T cells of 18% or greater, determined using the CSLEX mAb in a sample from the subject prior to immunotherapy, is indicative that the subject is responsive to the immunotherapy.

In certain embodiments of the methods and systems of the invention, a level of the CTLA-4 marker in Ki67+CD4+ T cells of 42% or greater, in a sample from the subject prior to and/or after the immunotherapy, is indicative that the subject is responsive to the immunotherapy.

In certain embodiments of the methods and systems of the invention, a level of the FoxP3 marker in Ki67+CD4+ T cells of 30% or greater, in a sample from the subject prior to immunotherapy, is indicative that the subject is responsive to the immunotherapy.

In certain embodiments of the methods and systems of the invention, a level of the CD15s marker in Ki67+CD4− CD8− T cells of 3% or greater, in a sample from the subject prior to immunotherapy, is indicative that the subject is responsive to the immunotherapy.

In certain embodiments of the methods and systems of the invention, a level of the Ki67+ marker in CD8+ T cells of 4% or less in a sample from the subject prior to immunotherapy, is indicative that the subject is responsive to the immunotherapy.

In certain embodiments of the methods and systems of the invention, the level of the marker is compared to the level of the markers in a non-responsive subject, and/or a predetermined level of the marker.

In certain embodiments, the methods and systems of the invention comprise determining the level of transcription of one or more of the markers.

In certain embodiments, the methods and systems of the invention comprise determining the level of the one or more markers in blood.

In certain embodiments, the methods and systems of the invention comprise determining the level of the one or more markers in a tumour biopsy.

In certain embodiments, the methods and systems of the invention comprise determining the level of the one or more markers in tumour infiltrating lymphocytes.

In certain embodiments, the methods and systems of the invention comprise use of a genetic characteristic of the cancer, and/or one or more genetic and/or clinical characteristics of the subject, to assess responsiveness of the subject to the immunotherapy.

In certain embodiments, the methods and systems of the invention are used to select a subject suitable for immune checkpoint inhibitor mono-immunotherapy and/or to select a subject suitable for a non-immune checkpoint inhibitory immunotherapy.

In certain embodiments of the methods and systems of the invention, the cancer is selected from one of melanoma, non-small cell lung cancer, mesothelioma, kidney cancer, head and neck cancer, Hodgkin Lymphoma, Merkel cell carcinoma, bladder cancer, triple negative breast cancer, oesophageal cancer, gastric cancer, squamous cell carcinoma of the skin, cervical cancer, a cancer with microsatellite instability and/or mismatch repair enzyme deficiency, hepatocellular carcinoma, and primary mediastinal large B-cell lymphoma, and other malignancies susceptible to therapeutic immune checkpoint blockade.

In certain embodiment, the method of the invention comprises using computer software executable by a processor to process data representative of the vascular and/or tumour associated level of one or more of the markers, and thereby provide a measure of the responsiveness of the subject to the immunotherapy.

In certain embodiments, the invention provides a method of treating a subject suffering from, or susceptible to, a cancer with an anti-PD1 immune checkpoint inhibitor immunotherapy (or a system for selecting a subject for a method), the method comprising identifying a subject responsive to the immune checkpoint inhibitor immunotherapy using the method or system as disclosed herein, and treating the subject so identified with the immune checkpoint inhibitor immunotherapy.

Accordingly, in a further embodiment, the present disclosure provides disclosure provide a method of determining responsiveness of a subject suffering from, or susceptible to, a cancer to anti-PD1 immune checkpoint inhibitor immunotherapy, the method comprising assessing the response of the subject to the immunotherapy on the basis of the vascular and/or tumour associated level in the subject of one or more of CD15s marker in CD71+CD8+ T cells, CTLA-4 marker in CD71+CD4+ T cells, FoxP3 marker in CD71+CD4+ T cells, CD15s marker in CD71+CD4− CD8− T cells, and CD71 marker in CD8+ T cells, wherein an increased level of the CD15s marker in the CD71+CD8+ T cells prior to the immunotherapy, an increased level of the CTLA-4 marker prior to and/or after the immunotherapy, an increased level of the FoxP3 marker prior to immunotherapy, an increased level of the CD15s marker in CD71+CD4− CD8− T cells prior to immunotherapy, and a decreased level of the CD71+ marker prior to immunotherapy, in the said T cells is indicative that the subject is responsive to the immunotherapy.

Certain embodiments of the present disclosure provide a method of determining responsiveness of a subject suffering from, or susceptible to, a cancer to anti-PD1 immune checkpoint inhibitor immunotherapy, the method comprising assessing the response of the subject to the immunotherapy on the basis of the level in the blood in the subject of one or more of CD15s marker in CD71+CD8+ T cells, CTLA-4 marker in CD71+CD4+ T cells, FoxP3 marker in CD71+CD4+ T cells, CD15s marker in CD71+CD4− CD8− T cells, and CD71 marker in CD8+ T cells, wherein an increased level of the CD15s marker in the CD71+CD8+ T cells prior to the immunotherapy, an increased level of the CTLA-4 marker prior to and/or after the immunotherapy, an increased level of the FoxP3 marker prior to immunotherapy, an increased level of the CD15s marker in CD71+CD4− CD8− T cells prior to immunotherapy, and a decreased level of the CD71+ marker prior to immunotherapy, in the said T cells is indicative that the subject is responsive to the immunotherapy.

Certain embodiments of the present disclosure provide a method of determining responsiveness of a subject suffering from, or susceptible to, a cancer to anti-PD1 immune checkpoint inhibitor immunotherapy, the method comprising assessing the response of the subject to the immunotherapy on the basis of the level in cells in a tumour in the subject of one or more of CD15s marker in CD71+CD8+ T cells, CTLA-4 marker in CD71+CD4+ T cells, FoxP3 marker in CD71+CD4+ T cells, CD15s marker in CD71+CD4− CD8− T cells, and CD71 marker in CD8+ T cells, wherein an increased level of the CD15s marker in the CD71+CD8+ T cells prior to the immunotherapy, an increased level of the CTLA-4 marker prior to and/or after the immunotherapy, an increased level of the FoxP3 marker prior to immunotherapy, an increased level of the CD15s marker in CD71+CD4− CD8− T cells prior to immunotherapy, and a decreased level of the CD71+ marker prior to immunotherapy, in the said T cells is indicative that the subject is responsive to the immunotherapy.

Certain embodiments of the present disclosure provide a method of determining responsiveness of a subject suffering from, or susceptible to, a cancer to anti-PD1 immune checkpoint inhibitor immunotherapy, the method comprising assessing the level in tumour infiltrating lymphocytes in the subject of one or more of CD15s marker in CD71+CD8+ T cells, CTLA-4 marker in CD71+CD4+ T cells, FoxP3 marker in CD71+CD4+ T cells, CD15s marker in CD71+CD4− CD8− T cells, and CD71 marker in CD8+ T cells, wherein an increased level of the CD15s marker in the CD71+CD8+ T cells prior to the immunotherapy, an increased level of the CTLA-4 marker prior to and/or after the immunotherapy, an increased level of the FoxP3 marker prior to immunotherapy, an increased level of the CD15s marker in CD71+CD4− CD8− T cells prior to immunotherapy, and a decreased level of the CD71+ marker prior to immunotherapy, in the said T cells is indicative that the subject is responsive to the immunotherapy.

Certain embodiments of the present disclosure provide a method of treating a subject suffering from, or susceptible to, a cancer with an anti-PD1 immune checkpoint inhibitor immunotherapy, the method comprising identifying a subject responsive to the immune checkpoint inhibitor immunotherapy using a method as described herein and treating the subject so identified with the immune checkpoint inhibitor immunotherapy.

Certain embodiments of the present disclosure provide a method of treating a subject suffering from, or susceptible to, a cancer with an anti-PD1 immune checkpoint inhibitor immunotherapy, the method comprising:
determining responsiveness of the subject to the immune checkpoint inhibitor immunotherapy on the basis of the vascular and/or tumour associated level of one or more of one or more of CD15s marker in CD71+CD8+ T cells, CTLA-4 marker in CD71+CD4+ T cells, FoxP3 marker in CD71+CD4+ T cells, CD15s marker in CD71+CD4− CD8− T cells, and CD71 marker in CD8+ T cells, wherein an increased level of the CD15s marker in the CD71+CD8+ T cells prior to the immunotherapy, an increased level of the CTLA-4 marker prior to and/or after the immunotherapy, an increased level of the FoxP3 marker prior to immunotherapy, an increased level of the CD15s marker in CD71+CD4− CD8− T cells prior to immunotherapy, and a decreased level of the CD71+ marker prior to immunotherapy, in the said T cells is indicative that the subject is responsive to the immunotherapy; and
treating the subject so determined to be responsive with the immunotherapy.

Certain embodiments of the present disclosure provide a kit for use in assessing responsiveness of a subject suffering from, or susceptible to, a cancer to anti-PD1 immune checkpoint inhibitor immunotherapy; the kit comprising one or more of an immunological reagent for detecting CD15s marker, an immunological reagent for detecting CTLA-4 marker, an immunological reagent for detecting FoxP3 marker, and an immunological reagent for detecting CD71 marker.

Certain embodiments of the present disclosure provide use of one or more of CD15s, CTLA-4, FoxP3 and CD71 as a marker to assess the responsiveness of a subject suffering from, or susceptible to, a cancer to anti-PD1 immune checkpoint inhibitor immunotherapy.

Certain embodiments of the present disclosure provide a method of assessing responsiveness of a subject suffering from, or susceptible to, a cancer to anti-PD1 immune checkpoint inhibitor immunotherapy, the method comprising:
receiving data representative of the vascular and/or tumour associated level of one or more of CD15s marker in CD71+CD8+ T cells, CTLA-4 marker in CD71+CD4+ T cells, FoxP3 marker in CD71+CD4+ T cells, CD15s marker in CD71+CD4− CD8− T cells, and CD71 marker in CD8+ T cells; and
processing the data to determine the responsiveness of the subject to the immunotherapy.

Certain embodiments of the present disclosure provide a system for assessing responsiveness of a subject suffering from, or susceptible to, a cancer to anti-PD1 immune checkpoint inhibitor immunotherapy, the system comprising:
a means for determining the level of one or more of CD15s marker in CD71+CD8+ T cells, CTLA-4 marker in CD71+CD4+ T cells, FoxP3 marker in CD71+CD4+ T cells, CD15s marker in CD71+CD4− CD8− T cells, and CD71 marker in CD8+ T cells, in a blood and/or tumour associated sample from a subject;
   a processor;
   a memory; and
   software resident in the memory accessible to the processor, the software comprising a series of instructions executable by the processor to process data representative of the vascular and/or tumour associated level of the one or more of the markers, and thereby provide a measure of the responsiveness of the subject to the immunotherapy.

Certain embodiments of the present disclosure provide a system for assessing responsiveness of a subject suffering from, or susceptible to, a cancer to anti-PD1 immune checkpoint inhibitor immunotherapy, the system comprising:
a flow cytometer;
a processor;
a memory; and
software resident in the memory accessible to the processor, the software comprising a series of instructions executable by the processor to process data received from the flow cytometer representative of the vascular and/or tumour associated level of the CD15s marker in CD71+CD8+ T cells, CTLA-4 marker in CD71+CD4+ T cells, FoxP3 marker in CD71+CD4+ T cells, CD15s marker in CD71+CD4− CD8− T cells, and CD71 marker in CD8+ T cells, and thereby provide a measure of the responsiveness of the subject to the immunotherapy.

In certain embodiments of the methods and systems of the invention, the CD71+CD8+ T cells comprise CD71+CD8+CD3+ T cells.

In certain embodiments of the methods and systems of the invention, the CD71+CD8+ T cells comprise CD71+CD8+CD3+CD28+ T cells

In certain embodiments of the methods and systems of the invention, the CD71+CD4+ T cells comprise CD71+CD4+CD8− T cells.

In certain embodiments of the methods and systems of the invention, the CD71+CD4+ T cells comprise CD71+CD4+CD3+ T cells.

In certain embodiments the method comprises use of one or more of FH6, HECA-452, CHO131, and CSLEX monoclonal antibodies to determine the level of the CD15s marker in CD71+CD8+ T cells.

As CD71 is believed to select for a relevant subset of Ki67+ cells, the percentage of CD71+ cells co-expressing CD15s maybe 1.5-fold, 2-fold, 2.5-fold, 3-fold, 3.5-fold, 4-fold, 4.5-fold, 5-fold, 5.5-fold, 6-fold, 6.5-fold, or 7-fold relative than the percentage of Ki67+ cells expressing CD15s.

Accordingly, in certain embodiments of the methods and systems of the invention, a level of the CD15s marker in CD71+CD8+ T cells of 2% or greater, 3% or greater 4% or greater, 5% or greater, 6% or greater, 7% or greater, 8% or greater, 9% or greater, 10% or greater, 11% or greater, 12% or greater, 13% or greater, or 14% or greater, determined using the FH6 mAb in a sample from the subject prior to immunotherapy, is indicative that the subject is responsive to the immunotherapy.

In certain embodiments of the methods and systems of the invention, a level of the CD15s marker in CD71+CD8+ T cells of 10% or greater, 15% or greater, 20% or greater, 25% or greater, 30% or greater, 35% or greater, 40% or greater, 45% or greater, or 50% or greater, determined using the HECA-452 mAb in a sample from the subject prior to immunotherapy, is indicative that the subject is responsive to the immunotherapy.

In certain embodiments of the methods and systems of the invention, a level of the CD15s marker in CD71+CD8+ T cells of 18% or greater, 27% or greater, 36% or greater, 45% or greater, 54% or greater, 63% or greater, 72% or greater, 81% or greater, or 90% or greater, determined using the CHO131 mAb in a sample from the subject prior to immunotherapy, is indicative that the subject is responsive to the immunotherapy.

In certain embodiments of the methods and systems of the invention, a level of the CD15s marker in CD71+CD8+ T cells of 18% or greater, 27% or greater, 36% or greater, 45% or greater, 54% or greater, 63% or greater, 72% or greater, 81% or greater, or 90% or greater, determined using the CSLEX mAb in a sample from the subject prior to immunotherapy, is indicative that the subject is responsive to the immunotherapy.

In certain embodiments of the methods and systems of the invention, a level of the CTLA-4 marker in CD71+CD4+ T cells of 42% or greater, 63% or greater, or 82% or greater, in a sample from the subject prior to and/or after the immunotherapy, is indicative that the subject is responsive to the immunotherapy.

In certain embodiments of the methods and systems of the invention, a level of the FoxP3 marker in CD71+CD4+ T cells of 30% or greater, 45% or greater, 60% or greater, 75% or greater, or 90% or greater, in a sample from the subject prior to immunotherapy, is indicative that the subject is responsive to the immunotherapy.

In certain embodiments of the methods and systems of the invention, a level of the CD15s marker in CD71+CD4− CD8− T cells of 3% or greater, 4.5% or greater, 6% or greater, 7.5% or greater, 9% or greater, 10.5% or greater, 12% or greater, 13.5% or greater, or 15% or greater, 16.5% or greater, 18% or greater, 19.5% or greater, 21% or greater, in a sample from the subject prior to immunotherapy, is indicative that the subject is responsive to the immunotherapy.

In certain embodiments of the methods and systems of the invention, a level of the CD71+ marker in CD8+ T cells of 4% or less, 2.5% or less, 2% or less, 1.6% % or less, 1.3% or less, 1.15% or less, 1% or less, 0.9% or less, 0.8% or less, 0.73% or less, 0.67 or less, 0.62% or less, or 0.57% or less, in a sample from the subject prior to immunotherapy, is indicative that the subject is responsive to the immunotherapy.

Other embodiments are described herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

For a better understanding of the present disclosure, and to show more clearly how the present disclosure may be carried into effect according to one or more embodiments thereof, reference will be made, by way of example, to the accompanying figures.

FIG. 1 shows PBMCs isolated from pre-treatment blood samples and subjected to immuno-staining and flow cytometry analysis. Following exclusion of dead cells (FVS575V+), CD8+ T cells were gated on the basis of a CD3+CD8+ phenotype. Within this gate, the percentage of cells staining positive for Ki67 was determined. Patients were identified as responders (complete/partial response) or non-responders (stable/progressive disease) by RECIST criteria, and the frequency of Ki67+ cells within the two populations was compared using the Mann-Whitney test. Each point represents a single patient and lines represent group means.

FIG. 2 shows PBMCs isolated from pre-treatment blood samples and subjected to immuno-staining and flow cytometry analysis. Following exclusion of dead cells (FVS575V+), proliferating CD8+ T cells were gated on the basis of a CD3+CD8+Ki67+ phenotype. Within this gate, the percentage of cells staining positive for CD15s using the HECA-452 mAb (also referred to as CLA; cutaneous lymphocyte antigen) was determined. Patients were identified as responders (complete/partial response) or non-responders (stable/progressive disease) by RECIST criteria, and the frequency of CD15s+ cells within the two populations was compared using the Mann-Whitney test. Each point represents a single patient and lines represent group means.

FIG. 3 shows PBMCs isolated from pre-treatment blood samples and subjected to flow cytometry staining and analysis. Following exclusion of dead cells (FVS575V+), proliferating CD8+ T cells were gated on the basis of a CD3+CD8+Ki67+ phenotype. Within this gate, the percentage of cells staining positive for CD15s using the FH6 mAb was determined. Patients were identified as responders (complete/partial response) or non-responders (stable/progressive disease) by RECIST criteria, and the frequency of CD15s+ cells within the two populations was compared using the Mann-Whitney test. Each point represents a single patient and lines represent group means.

FIG. 4 shows PBMCs isolated from pre-treatment blood samples and subjected to flow cytometry staining and analysis. Following exclusion of dead cells (FVS575V+), proliferating CD8+ T cells were gated on the basis of a CD3+CD8+Ki67+ phenotype. Within this gate, the percentage of cells staining positive for CD15s using the CHO131 mAb was determined. Patients were identified as responders (complete/partial response) or non-responders (stable/progressive disease) by RECIST criteria, and the frequency of CD15s+ cells within the two populations was compared using the Mann-Whitney test. Each point represents a single patient and lines represent group means.

FIG. 5 shows PBMCs isolated from pre-treatment blood samples and subjected to immuno-staining and flow cytometry analysis. Following exclusion of dead cells (FVS575V+), proliferating CD8+ T cells were gated on the basis of a CD3+CD8+Ki67+ phenotype. Within this gate, the percentage of cells staining positive for CD15s using the CSLEX mAb was determined. Patients were identified as responders (complete/partial response) or non-responders (stable/progressive disease) by RECIST criteria, and the frequency of CD15s+ cells within the two populations was compared using the Mann-Whitney test. Each point represents a single patient and lines represent group means.

FIG. 6 shows PBMCs isolated from pre-treatment blood samples and subjected to immuno-staining and flow cytometry analysis. Following exclusion of dead cells (FVS575V+), proliferating double negative T cells were gated on the basis of a CD3+CD4− CD8− Ki67+ phenotype. Within this gate, the percentage of cells staining positive for CD15s using the HECA-452 mAb was determined. Patients were identified as responders (complete/partial response) or non-responders (stable/progressive disease) by RECIST criteria, and the frequency of CD15s+ cells within the two populations were compared using the Mann-Whitney test. Each point represents a single patient and lines represent group means.

FIG. 7 shows PBMCs isolated from pre-treatment blood samples and subjected to immuno-staining and flow cytometry analysis. Following exclusion of dead cells (FVS575V+), proliferating CD4+ T cells were gated on the basis of a CD3+CD4+Ki67+ phenotype. Within this gate, the percentage of cells staining positive for CTLA-4 was determined. Patients were identified as responders (complete/partial response) or non-responders (stable/progressive disease) by RECIST criteria, and the frequency of CTLA-4+ cells within the two populations was compared using the Mann-Whitney test. Each point represents a single patient and lines represent group means.

FIG. 8 shows PBMCs isolated from pre-treatment blood samples and subjected to immuno-staining and flow cytometry analysis. Following exclusion of dead cells (FVS575V+), proliferating CD4+ T cells were gated on the basis of a CD3+CD4+Ki67+ phenotype. Within this gate, the percentage of cells staining positive for FoxP3 was determined. Patients were identified as responders (complete/partial response) or non-responders (stable/progressive disease) by RECIST criteria, and the frequency FoxP3+ cells within the two populations was compared using the Mann-Whitney test. Each point represents a single patient and lines represent group means.

FIG. 9 shows the results for an algorithm that was developed to predict whether individual patients responded to anti-PD1 therapy, on the basis of the flow cytometric analysis of their pre-treatment blood sample.

The tally of points (score) for each patient is shown. Patients scoring 3 or above were considered as predicted responders, while patients scoring 2 or below were considered as predicted non-responders.

FIG. 10a to FIG. 10c show a visual guide to flow cytometry data analysis. The examples illustrate the correct placement of discriminators (gates and quadrants) to accurately identify cells expressing key markers for the approach. The gates on the scatter plots on the left of FIGS. 10a and 10b show the correct placement for identifying Ki67+ cells (FIG. 10a) and CD15s+ cells (FIG. 10b) while the gates in the central column are too far left and the right column are too far right. FIG. 10c shows gating for FoxP3 (vertical axis) and CTLA-4 (horizontal axis) with appropriate gating on the scatter plots in the left column, gating too far left and down in the central column and gating too far right and up in the right column.

FIGS. 11a and 11b show the correlation of CD71 expression with Ki67 expression. FIG. 11a shows flow cytometry analysis of CD8+ PBMCs, co-stained for Ki67 (horizontal axis) and CD71 (vertical axis). FIG. 11b illustrates the portion of CD71 expressing cells that co-express Ki67 expression.

### DETAILED DESCRIPTION

The present disclosure relates to methods and products for determining the responsiveness of a subject suffering from, or susceptible to, a cancer to immune checkpoint inhibitor (ICI) immunotherapy.

The present disclosure is based, at least in part, on the recognition that distinct T cell subsets associate with clinical responsiveness to ICI drugs, and measuring the frequency of these subsets enables prediction of responsiveness prior to ICI treatment.

One or more embodiments of the present disclosure are directed to methods and products that have one or more of the following advantages: new and/or improved methods and products for assessing the responsiveness of a patient or subject to anti-PD1 immune checkpoint inhibitor therapy; new and/or improved methods and products for screening patients or subjects for responsiveness to anti-PD1 immune checkpoint inhibitor therapy; new and/or improved methods and products for identifying patients or subjects likely to respond to anti-PD1 immune checkpoint inhibitor therapy; new methods and products to assist with the selection of personalised treatment decisions for patients or subjects who may or may not respond to anti-PD1 therapy; new and/or improved methods and products for identifying patients or subjects who are not likely to respond to anti-PD1 immune checkpoint inhibitor therapy; new and/or improved methods and products for reducing the number of patients or subjects who may receive potentially toxic anti-PD1 immune checkpoint inhibitor therapy without the likelihood of therapeutic benefit; methods and products for identifying patients or subjects who should be treated with standard cancer treatments rather than anti-PD1 immune checkpoint therapy; methods and products for improving rates of response to anti-PD1 immune checkpoint inhibitor therapy; new and/or improved methods and products for measuring cells in blood or in tumour samples which are indicative of whether a patient or subject will respond to anti-PD1 immune checkpoint inhibitor therapy; new and/or improved methods and products for assessing responsiveness to anti-PD1 immune checkpoint inhibitor therapy before the therapy has begun; new and/or improved methods and products for assessing responsiveness to anti-PD1 immune checkpoint inhibitor therapy after the therapy has begun; to address one or more problems, and/or to provide one or more advantages, or to provide a commercial alternative. Other advantages of certain embodiments of the present disclosure are also disclosed herein.

Certain embodiments of the present disclosure provide a method of determining responsiveness of a subject suffering from, or susceptible to, a cancer to anti-PD1 immune checkpoint inhibitor immunotherapy.

In certain embodiments, the present disclosure provides a method of determining responsiveness of a subject suffering from, or susceptible to, a cancer to anti-PD1 immune checkpoint inhibitor immunotherapy, the method comprising assessing the response of the subject to the immunotherapy on the basis of the vascular and/or tumour associated level in the subject of one or more of CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells, wherein an increased level of the CD15s marker in the Ki67+CD8+ T cells prior to the immunotherapy, an increased level of the CTLA-4 marker prior to and/or after the immunotherapy, an increased level of the FoxP3 marker prior to immunotherapy, an increased level of the CD15s marker in Ki67+CD4− CD8− T cells prior to immunotherapy, and a decreased level of the Ki67+ marker prior to immunotherapy, in the said T cells is indicative that the subject is responsive to the immunotherapy.

The use of immune checkpoint inhibitors in treatment of cancers is described, for example, in “Immune Checkpoint Inhibitors in Cancer”; Fumito Ito and Marc S. Ernstoff—Authors, (2019) Elsevier Inc.

In certain embodiments, the method is used to assess responsiveness to anti-PD1 immune checkpoint inhibitor immunotherapy, to screen a subject for their responsiveness to anti-PD1 immune checkpoint inhibitor immunotherapy, and to identify a subject who will, or will not, respond to anti-PD1 immune checkpoint inhibitor immunotherapy.

In certain embodiments, the anti-PD1 immune checkpoint inhibitor immunotherapy comprises use of a monoclonal antibody directed against PD1. Examples include the inhibitors pembrolizumab or nivolumab.

In certain embodiments, the anti-PD1 immune checkpoint inhibitor immunotherapy comprises use of a monoclonal antibody directed against a ligand of the PD1 receptor. Examples include PD-L1 and the inhibitors avelumab or atezolizumab.

The cancer for treatment as described herein refers to a cancer that is susceptible to treatment with an immune checkpoint inhibitor.

In certain embodiments, the cancer comprises one of cancer selected from the group of a melanoma, a non-small cell lung cancer, a mesothelioma, a kidney cancer, a head and neck cancer, a Hodgkin Lymphoma, a Merkel cell carcinoma, a bladder cancer, a triple negative breast cancer, an oesophageal cancer, a gastric cancer, a squamous cell carcinoma of the skin, a cervical cancer, a cancer with microsatellite instability and/or mismatch repair enzyme deficiency, a hepatocellular carcinoma, a primary mediastinal large B-cell lymphoma, and other malignancies susceptible to therapeutic immune checkpoint blockade. Other types of cancers are contemplated.

In certain embodiments, the cancer is a melanoma, a Hodgkin lymphoma, a non-small cell lung cancer, and an urothelial cancer, and the treatment is with a checkpoint inhibitor that blocks PD1, such as nivolumab or pembrolizumab.

In certain embodiments, the cancer is a lung cancer, an urothelial cancer, or a lung cancer and the treatment is with a checkpoint inhibitors that blocks PD-L1, such as atezolizumab.

In certain embodiments, the immune checkpoint inhibitor immunotherapy comprises use of inhibitors to two or more separate targets, for example PD1 and PD-L1 or PD1/PD-L1 and CTLA-4.

In certain embodiments, the subject is suffering from a cancer. In certain embodiments, the subject is susceptible to a cancer.

In certain embodiments, the one or more markers are cell surface markers. Methods for detecting and assessing the level of cell surface markers are known in the art.

In certain embodiments, the one or more markers are intracellular markers. Methods for detecting and assessing the level of intracellular markers are known in the art.

Methods for identifying markers described herein are known in the art. For example, flow cytometry may be used to identify the cells using the appropriate agents for detecting the cell surface markers. Other methods are contemplated such as use of magnetic beads.

In certain embodiments, the Ki67+CD8+ T cells comprise Ki67+CD8+CD3+ T cells. Methods for identifying Ki67+CD8+CD3+ T cells are known in the art.

In certain embodiments, the Ki67+CD8+ T cells comprise Ki67+CD8+CD3+CD28+ T cells. Methods for identifying Ki67+CD8+CD3+CD28+ T cells are known in the art.

In certain embodiments, the Ki67+CD4+ T cells comprise Ki67+CD4+CD8− T cells.

In certain embodiments, the method the Ki67+CD4+ T cells comprise Ki67+CD4+CD3+ T cells.

In certain embodiments, the method comprises determining the level of one or more of the markers expressed by cells by flow cytometry.

In certain embodiments, the method comprises use of one or more of FH6, HECA-452, CHO131, and CSLEX monoclonal antibodies to determine the level of the CD15s marker on T cells, such as those described herein.

In certain embodiments, a level of the CD15s marker in Ki67+CD8+ T cells of 2% or greater, 3% or greater, 4% or greater, 5% or greater, 6% or greater determined using the FH6 mAb in a sample from the subject prior to immunotherapy is indicative that the subject is responsive to the immunotherapy. In some embodiments, a level of the CD15s marker in Ki67+CD8+ T cells of approximately 3.5% or greater, determined using the FH6 mAb in a sample from the subject prior to immunotherapy, is indicative that the subject is not responsive to the immunotherapy.

In certain embodiments, a level of the CD15s marker in Ki67+CD8+ T cells of 3% or greater, 4% or greater, 5% or greater, 6% or greater, or 7% or greater, determined using the FH6 mAb in a sample from the subject prior to immunotherapy is indicative that the subject is responsive to the immunotherapy. In some embodiments, a level of the CD15s marker in Ki67+CD8+ T cells of approximately 5.3% or greater, determined using the FH6 mAb in a sample from the subject prior to immunotherapy, is indicative that the subject is not responsive to the immunotherapy.

In certain embodiments, a level of the CD15s marker in Ki67+CD8+ T cells of 7% or less, 6% or less, 5% or less, 4% or less, or 3% or less determined using the FH6 mAb in a sample from the subject prior to immunotherapy is indicative that the subject is not responsive to the immunotherapy. In some embodiments, a level of the CD15s marker in Ki67+CD8+ T cells of approximately 5.3% or less, determined using the FH6 mAb in a sample from the subject prior to immunotherapy, is indicative that the subject is not responsive to the immunotherapy.

In certain embodiments, a level of the CD15s marker in Ki67+CD8+ T cells of 2% or less, 3% or less, 4% or less, 5% or less, or 6% or less determined using the FH6 mAb in a sample from the subject prior to immunotherapy is indicative that the subject is not responsive to the immunotherapy. In some embodiments, a level of the CD15s marker in Ki67+CD8+ T cells of approximately 3.5% or less, determined using the FH6 mAb in a sample from the subject prior to immunotherapy, is indicative that the subject is not responsive to the immunotherapy.

In certain embodiments, a level of the CD15s marker in Ki67+CD8+ T cells of 7% or greater, 8% or greater, 9% or greater, 10% or greater, 11% or greater, 12% or greater, or 13% or greater, determined using the HECA mAb in a sample from the subject prior to immunotherapy is indicative that the subject is responsive to the immunotherapy. In some embodiments, a level of the CD15s marker in Ki67+CD8+ T cells of approximately 10.3% or greater, determined using the HECA mAb in a sample from the subject prior to immunotherapy, is indicative that the subject is responsive to the immunotherapy.

In certain embodiments, a level of the CD15s marker in Ki67+CD8+ T cells of 17% or greater, 18% or greater, 19% or greater, 20% or greater, or 21% or greater, determined using the HECA mAb in a sample from the subject prior to immunotherapy is indicative that the subject is responsive to the immunotherapy. In some embodiments, a level of the CD15s marker in Ki67+CD8+ T cells of approximately 19.2% or greater, determined using the HECA mAb in a sample from the subject prior to immunotherapy, is indicative that the subject is responsive to the immunotherapy.

In certain embodiments, a level of the CD15s marker in Ki67+CD8+ T cells of 22% or less, 20% or less, 18% or less, 16% or less, or 14% or less, determined using the HECA mAb in a sample from the subject prior to immunotherapy, is indicative that the subject is not responsive to the immunotherapy. In some embodiments, a level of the CD15s marker in Ki67+CD8+ T cells of approximately 19.2% or less, determined using the HECA mAb in a sample from the subject prior to immunotherapy, is indicative that the subject is not responsive to the immunotherapy.

In certain embodiments, a level of the CD15s marker in Ki67+CD8+ T cells of 12% or less, 11% or less, 10% or less, 9% or less, or 8% or less, determined using the HECA mAb in a sample from the subject prior to immunotherapy, is indicative that the subject is not responsive to the immunotherapy. In some embodiments, a level of the CD15s marker in Ki67+CD8+ T cells of approximately 10.3% or less, determined using the HECA mAb in a sample from the subject prior to immunotherapy, is indicative that the subject is not responsive to the immunotherapy

In certain embodiments, a level of the CD15s marker in Ki67+CD8+ T cells of 17% or greater, 18% or greater, 19% or greater, 20% or greater, 21% or greater, or 22% or greater, determined using the CHO131 mAb in a sample from the subject prior to immunotherapy, is indicative that the subject is responsive to the immunotherapy. In certain embodiments, a level of the CD15s marker in Ki67+CD8+ T cells of approximately 19.6% or greater, determined using the CHO131 mAb in a sample from the subject prior to immunotherapy, is indicative that the subject is responsive to the immunotherapy.

In certain embodiments, a level of the CD15s marker in Ki67+CD8+ T cells of 26% or greater, 27% or greater, 28% or greater, 29% or greater, or 30% or greater, determined using the CHO131 mAb in a sample from the subject prior to immunotherapy, is indicative that the subject is responsive to the immunotherapy. In certain embodiments, a level of the CD15s marker in Ki67+CD8+ T cells of approximately 28.6% or greater, determined using the CHO131 mAb in a sample from the subject prior to immunotherapy, is indicative that the subject is responsive to the immunotherapy.

In certain embodiments, a level of the CD15s marker in Ki67+CD8+ T cells of 30% or less, 29% or less, 28% or less, 26% or less, or 25% or less, determined using the CHO131 mAb in a sample from the subject prior to immunotherapy, is indicative that the subject is not responsive to the immunotherapy. In certain embodiments a level of the CD15s marker in Ki67+CD8+ T cells of approximately 28.6% or less, determined using the CHO131 mAb in a sample from the subject prior to immunotherapy, is indicative that the subject is not responsive to the immunotherapy.

In certain embodiments, a level of the CD15s marker in Ki67+CD8+ T cells of 22% or less, 21% or less, 20% or less, 19% or less, or 18% or less, determined using the CHO131 mAb in a sample from the subject prior to immunotherapy, is indicative that the subject is not responsive to the immunotherapy. In certain embodiments a level of the CD15s marker in Ki67+CD8+ T cells of approximately 19.6% or less, determined using the CHO131 mAb in a sample from the subject prior to immunotherapy, is indicative that the subject is not responsive to the immunotherapy.

In certain embodiments, a level of the CD15s marker in Ki67+CD8+ T cells of 18% or greater, 19% or greater, 20% or greater, 21% or greater, or 22% or greater, determined using the CSLEX mAb in a sample from the subject prior to immunotherapy, is indicative that the subject is responsive to the immunotherapy. In certain embodiments, a level of the CD15s marker in Ki67+CD8+ T cells of approximately 20.2% or greater, determined using the CSLEX mAb in a sample from the subject prior to immunotherapy, is indicative that the subject is responsive to the immunotherapy.

In certain embodiments, a level of the CD15s marker in Ki67+CD8+ T cells of 26% or greater, 27% or greater, 28% or greater, 29% or greater, or 30% or greater, determined using the CSLEX mAb in a sample from the subject prior to immunotherapy, is indicative that the subject is responsive to the immunotherapy. In certain embodiments, a level of the CD15s marker in Ki67+CD8+ T cells of approximately 28.8% or greater, determined using the CSLEX mAb in a sample from the subject prior to immunotherapy, is indicative that the subject is responsive to the immunotherapy.

In certain embodiments, a level of the CD15s marker in Ki67+CD8+ T cells of 30% or less, 29% or less, 28% or less, 27% or less, or 26% or less, determined using the CSLEX mAb in a sample from the subject prior to immunotherapy, is indicative that the subject is not responsive to the immunotherapy. In certain embodiments, a level of the CD15s marker in Ki67+CD8+ T cells of approximately 28.8% or less, determined using the CSLEX mAb in a sample from the subject prior to immunotherapy, is indicative that the subject is not responsive not to the immunotherapy.

In certain embodiments, a level of the CD15s marker in Ki67+CD8+ T cells of 22% or less, 21% or less, 20% or less, 19% or less, or 18% or less, determined using the CSLEX mAb in a sample from the subject prior to immunotherapy, is indicative that the subject is not responsive to the immunotherapy. In certain embodiments, a level of the CD15s marker in Ki67+CD8+ T cells of approximately 20.2% or less, determined using the CSLEX mAb in a sample from the subject prior to immunotherapy, is indicative that the subject is not responsive not to the immunotherapy.

In certain embodiments, a level of the CTLA-4 marker in Ki67+CD4+ T cells of 42% or greater in a sample from the subject prior to and/or after the immunotherapy is indicative that the subject is responsive to the immunotherapy.

In certain embodiments, a level of the CTLA-4 marker in Ki67+CD4+ T cells of 48% or greater, 49% or greater, 50% or greater, 51% or greater, or 52% or greater, in a sample from the subject prior to and/or after the immunotherapy is indicative that the subject is responsive to the immunotherapy. In certain embodiments, a level of the CTLA-4 marker in Ki67+CD4+ T cells of approximately 50.1% or greater, in a sample from the subject prior to and/or after the immunotherapy is indicative that the subject is responsive to the immunotherapy.

In certain embodiments, a level of the CTLA-4 marker in Ki67+CD4+ T cells of 58% or greater, 59% or greater, 60% or greater, 61% or greater, or 62% or greater, in a sample from the subject prior to and/or after the immunotherapy is indicative that the subject is responsive to the immunotherapy. In certain embodiments, a level of the CTLA-4 marker in Ki67+CD4+ T cells of approximately 60.4% or greater, in a sample from the subject prior to and/or after the immunotherapy is indicative that the subject is responsive to the immunotherapy.

In certain embodiments, a level of the CTLA-4 marker in Ki67+CD4+ T cells of 48% or less, 49% or less, 50% or less, 51% or less, or 52% or less, in a sample from the subject prior to and/or after the immunotherapy is indicative that the subject is not responsive to the immunotherapy. In certain embodiments, a level of the CTLA-4 marker in Ki67+CD4+ T cells of approximately 50.1% or less, in a sample from the subject prior to and/or after the immunotherapy is indicative that the subject is not responsive to the immunotherapy.

In certain embodiments, a level of the CTLA-4 marker in Ki67+CD4+ T cells of 58% or less, 59% or less, 60% or less, 61% or less, or 62% or less, in a sample from the subject prior to and/or after the immunotherapy is indicative that the subject is not responsive to the immunotherapy. In certain embodiments, a level of the CTLA-4 marker in Ki67+CD4+ T cells of approximately 60.4% or less, in a sample from the subject prior to and/or after the immunotherapy is indicative that the subject is not responsive to the immunotherapy.

In certain embodiments, a level of the FoxP3 marker in Ki67+CD4+ T cells of 28% or greater, 29% or greater, 30% or greater, 31% or greater, or 32% or greater in a sample from the subject prior to immunotherapy is indicative that the subject is responsive to the immunotherapy. In certain embodiments, a level of the FoxP3 marker in Ki67+CD4+ T cells of approximately 29.4% or greater in a sample from the subject prior to immunotherapy is indicative that the subject is responsive to the immunotherapy.

In certain embodiments, a level of the FoxP3 marker in Ki67+CD4+ T cells of 34% or greater, 35% or greater, 36% or greater, 37% or greater, or 38% or greater in a sample from the subject prior to immunotherapy is indicative that the subject is responsive to the immunotherapy. In certain embodiments, a level of the FoxP3 marker in Ki67+CD4+ T cells of approximately 36.9% or greater in a sample from the subject prior to immunotherapy is indicative that the subject is responsive to the immunotherapy.

In certain embodiments, a level of the FoxP3 marker in Ki67+CD4+ T cells of 28% or less, 29% or less, 30% or less, 31% or less, or 32% or less in a sample from the subject prior to immunotherapy is indicative that the subject is not responsive to the immunotherapy. In certain embodiments, a level of the FoxP3 marker in Ki67+CD4+ T cells of approximately 29.4% or less in a sample from the subject prior to immunotherapy is indicative that the subject is not responsive to the immunotherapy.

In certain embodiments, a level of the FoxP3 marker in Ki67+CD4+ T cells of 34% or less, 35% or less, 36% or less, 37% or less, or 38% or less in a sample from the subject prior to immunotherapy is indicative that the subject is not responsive to the immunotherapy. In certain embodiments, a level of the FoxP3 marker in Ki67+CD4+ T cells of approximately 36.9% or less in a sample from the subject prior to immunotherapy is indicative that the subject is not responsive to the immunotherapy.

In certain embodiments, a level of the CD15s marker in Ki67+CD4− CD8− T cells of 1% or greater, 2% or greater, 3% or greater, 4% or greater, or 5% or greater, in a sample from the subject prior to immunotherapy is indicative that the subject is responsive to the immunotherapy. In certain embodiments, a level of the CD15s marker in Ki67+CD4− CD8− T cells of approximately 3.2% or greater, in a sample from the subject prior to immunotherapy is indicative that the subject is responsive to the immunotherapy.

In certain embodiments, a level of the CD15s marker in Ki67+CD4− CD8− T cells of 2% or greater, 3% or greater, 4% or greater, 5% or greater, or 6% or greater, in a sample from the subject prior to immunotherapy is indicative that the subject is responsive to the immunotherapy. In certain embodiments, a level of the CD15s marker in Ki67+CD4− CD8− T cells of approximately 4.4% or greater, in a sample from the subject prior to immunotherapy is indicative that the subject is responsive to the immunotherapy.

In certain embodiments, a level of the CD15s marker in Ki67+CD4− CD8− T cells of 5% or less, 4% or less, 3% or less, 2% or less or 1% or less, in a sample from the subject prior to immunotherapy is indicative that the subject is not responsive to the immunotherapy. In certain embodiments, a level of the CD15s marker in Ki67+CD4− CD8− T cells of approximately 3.2% or less, in a sample from the subject prior to immunotherapy is indicative that the subject is not responsive to the immunotherapy.

In certain embodiments, a level of the CD15s marker in Ki67+CD4− CD8− T cells of 6% or less, 5% or less, 4% or less, 3% or less or 2% or less, in a sample from the subject prior to immunotherapy is indicative that the subject is not responsive to the immunotherapy. In certain embodiments, a level of the CD15s marker in Ki67+CD4− CD8− T cells of approximately 4.4% or less, in a sample from the subject prior to immunotherapy is indicative that the subject is not responsive to the immunotherapy.

In certain embodiments, a level of the Ki67+ marker in CD8+ T cells of 1.5% or greater, or 2.8% or greater, in a sample from the subject prior to immunotherapy is indicative that the subject is not responsive to the immunotherapy.

In certain embodiments, a level of the Ki67+ marker in CD8+ T cells of 4% or greater, 3.5% or greater, 3.0% or greater, 2.5% or greater, 2.0% or greater, 1% or greater or 0.5% or greater, in a sample from the subject prior to immunotherapy is indicative that the subject is not responsive to the immunotherapy.

In certain embodiments, a level of the Ki67+ marker in CD8+ T cells of 1.5% or less in a sample from the subject prior to immunotherapy is indicative that the subject is responsive to the immunotherapy.

In certain embodiments, a level of the Ki67+ marker in CD8+ T cells of 1.5% or less, or 2.8% or less, in a sample from the subject prior to immunotherapy is indicative that the subject is responsive to the immunotherapy.

In certain embodiments, a level of the Ki67+ marker in CD8+ T cells of 4% or less, 3.5% or less, 3.0% or less, 2.5% or less, 2.0% or less, 1% or less, or 0.5% or less, in a sample from the subject prior to immunotherapy is indicative that the subject is responsive to the immunotherapy.

In certain embodiments, the method comprises determining the level, or proportion of cells expressing, of one or more of the markers by a method involving cell sorting. In certain embodiments, the method comprises determining the level of one or more of the markers by flow cytometry. Methods for using flow cytometry to determine the level of a marker are known in the art. Other methods are contemplated, such as other immunological detection methods. For example, immunocapture or immunosorbent methods may be used.

Methods for cell sorting are known in the art, for example as described in “Current Protocols in Cytometry” ed. J. Paul Robinson et al. (1999-2016), John Wiley & Sons.

In certain embodiments, the level of the marker is compared to the level of the marker(s) in a non-responsive subject, and/or a pre-determined level of the marker(s).

In certain embodiments, the level of the one or more markers (indicative of responsiveness to anti-PD1 ICI therapy) is greater than the median or mean level of the marker in non-responsive subjected. In some embodiments, the level of the one or more markers (indicative of responsiveness to anti-PD1 ICI therapy) is greater than the 50^{th} percentile, 60^{th} percentile, 70^{th} percentile, 75^{th} percentile, 80^{th} percentile, 85^{th} percentile, 90^{th} percentile, or 95^{th} percentile of non-responders.

In certain embodiments, the method comprises determining the level of transcription of one or more of the markers. Methods for determining the level of transcription are for example described in Sambrook et al. Molecular Cloning: A Laboratory Manual (4th ed., Cold Spring Harbor Laboratory Press, Cold Spring Harbor, N.Y. (2012); and Ausubel et al Current Protocols in Molecular Biology (2012) John Wiley & Sons.

In certain embodiments, the method comprises use of an immunological reagent to detect one or more of the markers. In certain embodiments, the immunological reagent comprises an antibody or an antigen binding part thereof.

The term “antibody” as used herein refers to an immunoglobulin molecule with the ability to bind an antigenic region of another molecule, and includes monoclonal antibodies, multivalent antibodies, chimeric antibodies, multispecific antibodies, diabodies and fragments/parts of an immunoglobulin molecule or combinations thereof that have the ability to bind to the antigenic region of another molecule with the desired affinity. Antigen binding fragments/parts of an antibody include a Fab, a Fab′, a F(ab′)₂, Fv, a single-chain antibody (scFv), a single domain antibody (sdAb) or a polypeptide that contains at least a portion of an immunoglobulin (or a variant of an immunoglobulin) that is sufficient to confer specific antigen binding, such as a molecule including one or more Complementarity Determining Regions (CDRs) from an antibody that binds to one or more of the markers.

In certain embodiments, the immunological reagent comprises a monoclonal antibody, or an antigen binding part thereof. Antibodies and antigen binding parts thereof useful for detection of the markers described herein thereof are known in the art or may be obtained commercially. Preferably, such antibodies will include at least one associated reporter, such as a fluorophore.

In certain embodiments, the method comprises determining the level of the one or more markers by immunostaining. Methods and reagents for performing and using immunostaining are known in the art.

In certain embodiments, the method comprises the use of reagents for detecting cell surface markers. In certain embodiments, the method comprises the use of reagent for detecting one or more intracellular markers. Methods for using reagents to detect cell surface markers or intracellular markers are known in the art.

In certain embodiments, the method comprises determining the level of the one or more markers in a sample of one or more of blood, or serum, or plasma, or an extract or derivative of any of the aforementioned.

In certain embodiments, the method comprises determining the level of the one or more markers in a tumour. In certain embodiments, the method comprises determining the level of the one or more markers in a tumour biopsy.

In certain embodiments, the method comprises obtaining a sample from the subject. In certain embodiments, the sample comprises a blood sample. In certain embodiments, the sample comprises a tissue sample. In certain embodiments, the sample comprises a biopsy. In certain embodiments, the sample comprises an extract or derivative of a tissue sample or a biopsy.

In certain embodiments, the sample comprises one or more cells isolated from a tumour (such as tumour infiltrating lymphocytes), and/or one or more cells isolated from a region surrounding a tumour.

In certain embodiments, the method comprises assessing isolated cells, such as isolated peripheral blood mononuclear cells. In certain embodiments, the method comprises assessing cells in situ, such as by assessing TILs by immunostaining in a tumour biopsy.

In certain embodiments, the vascular level of the one or more markers comprises the level of the one or more markers in cells in blood. In certain embodiments, the method comprises determining the level of the one or more markers in blood cells. Methods for obtaining a blood sample from a subject and processing the blood for analysis are known in the art.

In certain embodiments, the method comprises isolating peripheral blood mononuclear cells (PBMCs) and analysing the level of the one or more markers in those cells.

In certain embodiments, the method comprises isolating T cells. Methods for isolating T cells are known in the art.

In certain embodiments, the vascular level of the one or more markers comprises the level of the one or more markers in a sample or biopsy from a vein, an artery or one or more capillaries.

In certain embodiments, the tumour associated level of the one or more markers comprises the level of the one or more markers in a sample from a tumour, a tumour biopsy and/or in tumour infiltrating lymphocytes.

In certain embodiments, the method comprises determining the level of the one or more markers in a sample from a tumour, a tumour biopsy and/or in tumour infiltrating lymphocytes.

In certain embodiments, the method comprises determining the level of the one or more markers in tumour infiltrating lymphocytes. In certain embodiments, the method comprises determining the level of tumour infiltrating lymphocytes in a tumour, in a tumour sample or in a tumour biopsy.

In certain embodiments, the method further comprises use of a genetic characteristic of the cancer, and/or one or more genetic and/or clinical characteristics of the subject, to assess responsiveness of the subject to the immunotherapy. Examples include tumour mutation burden, tumour or immune cell PDL1 expression, and tumour inflammation gene expression.

In certain embodiments, the method is used to select a subject suitable for anti-PD1 immune checkpoint inhibitor immunotherapy. In certain embodiments, the method is used to select a subject suitable for anti-PD1 immune checkpoint inhibitor mono-immunotherapy. In certain embodiments, the method is used to select a subject that is not suitable for anti-PD1 immune checkpoint inhibitory immunotherapy. In certain embodiments, the method is used to select a subject suitable for therapies alternative to immune checkpoint inhibitory immunotherapy.

In certain embodiments, the method is used to assess the responsiveness of the subject to immunotherapy before treatment has started. In certain embodiments, the method is used to assess the responsiveness of the subject to immunotherapy after treatment has started. In certain embodiments, the method is used to assess the responsiveness of the subject to immunotherapy before and after treatment has started.

In certain embodiments, the method comprises producing data representative of the vascular and/or tumour associated level of one or more of the markers in specified cells. The data may for example comprise the level of expression or expression in a proportion of cells.

In certain embodiments, the method comprises the use of an algorithm to assess the responsiveness of the subject to immunotherapy. Methods are known in the art for processing data to assess a particular characteristic using an algorithm.

In certain embodiments, the data representative of the vascular and/or tumour associated level of one or more of the markers is used in an algorithm to assess the responsiveness of the subject to immunotherapy.

In certain embodiments, the method comprises using computer software executable by a processor to process data representative of the vascular and/or tumour associated level of one or more of the markers, and thereby provide a measure of the responsiveness of the subject to the immunotherapy.

In certain embodiments, the method comprises use of the data representative of the vascular and/or tumour associated level of one or more of the markers to produce a score, or a stratification level, for a subject likely to be responsive to the immune checkpoint inhibitor immunotherapy.

In certain embodiments, the present disclosure provides a method of determining responsiveness of a subject suffering from, or susceptible to, a cancer to anti-PD1 immune checkpoint inhibitor immunotherapy, the method comprising assessing the response of the subject to the immunotherapy on the basis of the level in the blood in the subject of one or more of CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells, wherein an increased level of the CD15s marker in the Ki67+CD8+ T cells prior to the immunotherapy, an increased level of the CTLA-4 marker prior to and/or after the immunotherapy, an increased level of the FoxP3 marker prior to immunotherapy, an increased level of the CD15s marker in Ki67+CD4− CD8− T cells prior to immunotherapy, and a decreased level of the Ki67+ marker prior to immunotherapy, in the said T cells is indicative that the subject is responsive to the immunotherapy.

In certain embodiments, the present disclosure provides a method of determining responsiveness of a subject suffering from, or susceptible to, a cancer to anti-PD1 immune checkpoint inhibitor immunotherapy, the method comprising assessing the response of the subject to the immunotherapy on the basis of the level in cells in a tumour in the subject of one or more of CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells, wherein an increased level of the CD15s marker in the Ki67+CD8+ T cells prior to the immunotherapy, an increased level of the CTLA-4 marker prior to and/or after the immunotherapy, an increased level of the FoxP3 marker prior to immunotherapy, an increased level of the CD15s marker in Ki67+CD4− CD8− T cells prior to immunotherapy, and a decreased level of the Ki67+ marker prior to immunotherapy, in the said T cells is indicative that the subject is responsive to the immunotherapy.

In certain embodiments, the present disclosure provides a method of determining responsiveness of a subject suffering from, or susceptible to, a cancer to anti-PD1 immune checkpoint inhibitor immunotherapy, the method comprising assessing the response of the subject to the immunotherapy on the basis of the level in tumour infiltrating lymphocytes in the subject of one or more of CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells, wherein an increased level of the CD15s marker in the Ki67+CD8+ T cells prior to the immunotherapy, an increased level of the CTLA-4 marker prior to and/or after the immunotherapy, an increased level of the FoxP3 marker prior to immunotherapy, an increased level of the CD15s marker in Ki67+CD4− CD8− T cells prior to immunotherapy, and a decreased level of the Ki67+ marker prior to immunotherapy, in the said T cells is indicative that the subject is responsive to the immunotherapy.

Certain embodiments of the present disclosure provide a method of treating a subject suffering from, or susceptible to, a cancer with a anti-PD1 immune checkpoint inhibitor.

In certain embodiments, the present disclosure provides a method of treating a subject suffering from, or susceptible to, a cancer with anti-PD1 immune checkpoint inhibitor immunotherapy, the method comprising identifying a subject responsive to the anti-PD1 immune checkpoint inhibitor immunotherapy using a method as described herein and treating an identified subject with an immune checkpoint inhibitor.

Examples of anti-PD1 immune checkpoint inhibitors for therapy are as described herein, although the use of other inhibitors is also contemplated.

In certain embodiments, the present disclosure provides a method of treating a subject suffering from, or susceptible to, a cancer with an anti-PD1 immune checkpoint inhibitor immunotherapy, the method comprising:
determining responsiveness of the subject to the immune checkpoint inhibitor immunotherapy on the basis of the vascular and/or tumour associated level of one or more of one or more of CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells, wherein an increased level of the CD15s marker in the Ki67+CD8+ T cells prior to the immunotherapy, an increased level of the CTLA-4 marker prior to and/or after the immunotherapy, an increased level of the FoxP3 marker prior to immunotherapy, an increased level of the CD15s marker in Ki67+CD4− CD8− T cells prior to immunotherapy, and a decreased level of the Ki67+ marker prior to immunotherapy, in the said T cells is indicative that the subject is responsive to the immunotherapy; and
treating the subject so determined to be responsive with the immunotherapy.

Methods for assessing the responsiveness of a subject to treatment are known in the art. For examples assessing tumour progression, regression, size, volume or markers of tumour burden (for examples prostate-specific antigen levels in blood).

Certain embodiments of the present disclosure provide a kit.

In certain embodiments, the present disclosure provides a kit for use in a method as described herein. In certain embodiments, the present disclosure provides a kit for performing a method as described herein.

Typically, a kit contains one or more reagents, one or more controls, and instructions for performing the method. Controls include on or more of positive controls such as cell expressing the designated marker, negative controls not expressing the designate marker, reference markers, and markers for normalising or correcting for expression.

In certain embodiments, the present disclosure provides a kit for use in assessing responsiveness of a subject suffering from, or susceptible to, a cancer to anti-PD1 immune checkpoint inhibitor immunotherapy; the kit comprising one or more of an immunological reagent for detecting CD15s, an immunological reagent for detecting CTLA-4, an immunological reagent for detecting FoxP3, and an immunological reagent for detecting Ki67 or CD71. Immunological reagents for detecting the aforementioned markers are described herein.

In certain embodiments, the kit further comprises one or more of an immunological reagent for detecting Ki67 or CD71, an immunological reagent for detecting CD8, an immunological reagent for detecting CD4, an immunological reagent for detecting CD3, and an immunological reagent for detecting CD28.

Certain embodiments of the present disclosure provide use of a marker as described herein for assessing the responsiveness of a subject suffering from, or susceptible to, a cancer to anti-PD1 immune checkpoint inhibitor immunotherapy.

In certain embodiments, the present disclosure provides use of one or more of CD15s, CTLA-4, FoxP3, and one or more of CD71 and Ki67 as a marker for assessing the responsiveness of a subject suffering from, or susceptible to, a cancer to anti-PD1 immune checkpoint inhibitor immunotherapy.

In certain embodiments, the present disclosure provides a method of assessing responsiveness of a subject suffering from, or susceptible to, a cancer to anti-PD1 immune checkpoint inhibitor immunotherapy, the method comprising:
receiving data representative of the vascular and/or tumour associated level of one or more of CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells; and
processing the data to determine the responsiveness of the subject to the immunotherapy.

Certain embodiments of the present disclosure provide computer software.

In certain embodiments, the present disclosure provides software for use with a computer comprising a processor and memory for storing the software, the software comprising a series of instructions executable by the processor to carry out a method as described herein.

In certain embodiments, the present disclosure provides software for use with a computer comprising a processor and memory for storing the software, the software comprising a series of instructions executable by the processor to assess the responsiveness of a subject to anti-PD1 immune checkpoint inhibitor immunotherapy using a method as described herein.

In certain embodiments, the present disclosure provides software for assessing the responsiveness of a subject to anti-PD1 immune checkpoint inhibitor immunotherapy, the software for use with a computer comprising a processor and memory for storing the software, the software comprising a series of instructions executable by the processor to carry out a method as described herein.

In certain embodiments, the software comprises instructions to process data representative of the one or more markers using an algorithm to assess the responsiveness of a subject to anti-PD1 immune checkpoint inhibitor immunotherapy.

Certain embodiments of the present disclosure provide a system for assessing responsiveness of a subject suffering from, or susceptible to, a cancer to anti-PD1 immune checkpoint inhibitor immunotherapy.

In certain embodiments, the present disclosure provides a system for assessing responsiveness of a subject suffering from, or susceptible to, a cancer to anti-PD1 immune checkpoint inhibitor immunotherapy, the system using a method as described herein to assess the responsiveness of a subject suffering from, or susceptible to, a cancer to the immune checkpoint inhibitor immunotherapy.

In certain embodiments, the present disclosure provides a system for assessing responsiveness of a subject suffering from, or susceptible to, a cancer to anti-PD1 immune checkpoint inhibitor immunotherapy, the system comprising a flow cytometer;
a processor;
a memory; and
software resident in the memory accessible to the processor, the software comprising a series of instructions executable by the processor to carry out a method as described herein.

Methods for determining the level of one or more markers are described herein. In certain embodiments, the means for determining the level of the one or more markers comprises a flow cytometer. Other methods are contemplated.

In certain embodiments, the present disclosure provides a system for assessing responsiveness of a subject suffering from, or susceptible to, a cancer to anti-PD1 immune checkpoint inhibitor immunotherapy, the system comprising
a means for determining the level of one or more of CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells, in a blood and/or tumour associated sample from a subject;
a processor;
a memory; and
software resident in the memory accessible to the processor, the software comprising a series of instructions executable by the processor to process data representative of the vascular and/or tumour associated level of the one or more of the markers, and thereby provide a measure of the responsiveness of the subject to the immunotherapy.

In certain embodiments, the present disclosure provides a system for assessing responsiveness of a subject suffering from, or susceptible to, a cancer to anti-PD1 immune checkpoint inhibitor immunotherapy, the system comprising
a flow cytometer;
a processor;
a memory; and
software resident in the memory accessible to the processor, the software comprising a series of instructions executable by the processor to process data received from the flow cytometer representative of the vascular and/or tumour associated level of the CD15s marker in Ki67+CD8+ T cells, CTLA-4 marker in Ki67+CD4+ T cells, FoxP3 marker in Ki67+CD4+ T cells, CD15s marker in Ki67+CD4− CD8− T cells, and Ki67 marker in CD8+ T cells, and thereby provide a measure of the responsiveness of the subject to the immunotherapy.

Statistical methods for analysis of biomarkers are known in the art, for example as described in Grund and Sabin (2010) Curr. Opin. HIV AIDS 5(6): 473-479. Algorithms based on the statistical analysis may be developed, including software employing the algorithms to determining responsiveness of a subject to therapy.

Standard techniques and equipment may be used for cell isolation, cell processing, immunological detection, flow cytometry, recombinant DNA technology, molecular biology and enzymatic reactions. The foregoing techniques and procedures may be generally performed according to methods known in the art and/or as commercially available, and are as described for example in Sambrook et al. Molecular Cloning: A Laboratory Manual (4th ed., Cold Spring Harbor Laboratory Press, Cold Spring Harbor, N.Y. (2012); and Ausubel et al Current Protocols in Molecular Biology (2012) John Wiley & Sons, both of which are hereby incorporated by reference.

The present disclosure is further described by the following examples. It is to be understood that the following description is for the purpose of describing particular embodiments only and is not intended to be limiting with respect to the above description

### Example 1—Assessing Responsiveness to Immune Checkpoint Inhibitor Therapy

The marker frequency on T cell populations isolated from patients undergoing immunotherapy using Immune Checkpoint Inhibitor (ICI) therapy were assessed to identify candidate markers of responsiveness to ICI therapy.

### Materials and Methods

Blood samples were collected from patients with metastatic melanoma about to begin treatment at the Royal Adelaide Hospital or Peter MacCallum Cancer Centre with single agent pembrolizumab or nivolumab, according to standard regimens. A blood specimen was collected immediately prior to the first drug infusion, or occasionally up to 1 week prior. Blood was collected into sterile heparanised tubes, and stored at room temperature until processing. Processing was generally conducted within 3 hours, or occasionally after overnight storage (≤16 hrs).

Blood was diluted with an equal volume of phosphate buffered saline (PBS) and the peripheral blood mononuclear cells (PBMCs) were isolated by centrifugation on Lymphoprep at 700×g for 20-30 minutes at room temperature. PBMCs were washed twice with PBS and then cryopreserved by resuspending in fetal bovine serum (FBS) containing 10% dimethyl sulphoxide (DMSO), aliquoting into cryovials and placing into a freezing container (either Mr Frosty or CoolCell) which was then placed into a −80° C. freezer for a minimum of 16 hr. Vials of cryopreserved cells were transferred to long-term storage in the vapour phase of liquid nitrogen until analysis.

To prepare samples of cryopreserved PBMCs for analysis, the vial contents were thawed rapidly in a 37° C. water bath, washed with PBS and resuspended in staining buffer (PBS+0.04% sodium azide+1% bovine serum albumin) for staining with antibodies. This same buffer was used to wash cells after each antibody incubation. Cells were incubated with fixable viability stain FVS575V (BD Biosciences) and washed, and then incubated with fluorochrome-conjugated antibodies to surface antigens (with the exception of the anti-CD15s clone FH6) and washed again. Cells were then fixed, permeabilised and washed using the eBioscience FoxP3 buffer set (Life Technologies) according to the manufacturer's instructions, and subsequently incubated with antibodies to Ki67 and, if required, CTLA-4, followed by washing. If required, cells were then incubated with anti-CD15s monoclonal antibodies (see Table 1) and washed (the FH6 antibody was found to be sensitive to the prior fixation/permeabilisation steps and optimal detection was obtained by adding this antibody as a final step). Flow cytometric analysis was performed on a BD LSR Fortessa flow cytometer. The antibodies were all commercially available. Raw flow cytometry data files were analysed using FCS Express (De Novo Software). Statistical analysis was conducted using GraphPad Prism; p values represent results of Mann-Whitney tests.

Best response to treatment at any time was determined for each patient on the basis of radiological measurement of tumour volume, according to the Response Evaluation Criteria in Solid Tumours (RECIST), v1.1. Other relevant demographic and clinical data were obtained from patient medical records.

TABLE 1
Details of antibodies used in flow cytometric analysis

| Specificity | Conjugate | Clone | Supplier |
|---|---|---|---|
| CD3 | BUV395 | SK7 | BD |
| CD4 | BV510 | SK3 | BD |
| CD8 | BV786 | SK1 | BD |
| Ki67 | BV421 | B56 | BD |
| CD15s (dimeric) | PE | FH6 | BioLegend |
| CD15s (conventional) | BV510 | CSLEX | BD |
| CD15s (conventional | AF647 | HECA-452 | BD |
| or sulphated) | | | |
| CD15s (conventional, | AF488 | CHO131 | R&D Systems |
| linked to core 2 O- | | | |
| glycan) | | | |
| CTLA4 | PECy7 | 14D3 | Life Technologies |
| FoxP3 | PerCP-Cy5.5 | PCH101 | Life Technologies |

### Results

Flow cytometry was used to measure the frequency of T cell subsets in blood samples collected from melanoma patients just prior to beginning therapy with an anti-PD1 agent (pembrolizumab or nivolumab). In addition, each patient's best response to therapy (after at least 6 months' follow-up) was assessed using RECIST criteria, and this information used to divide the patients into two groups: responders (complete or partial response) and non-responders (stable or progressive disease).

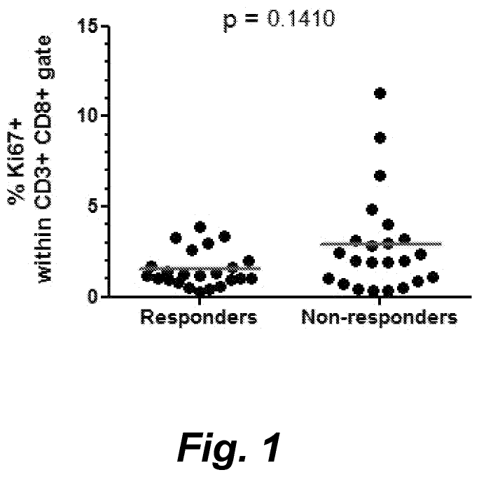

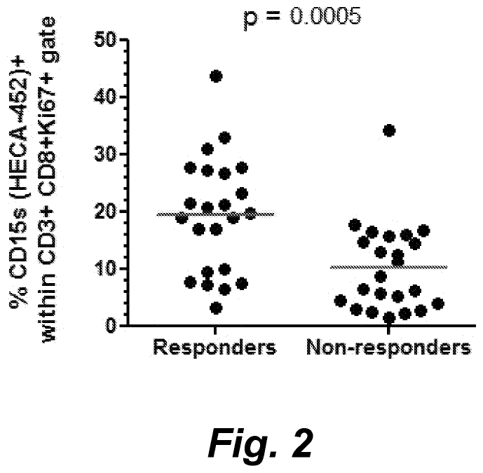

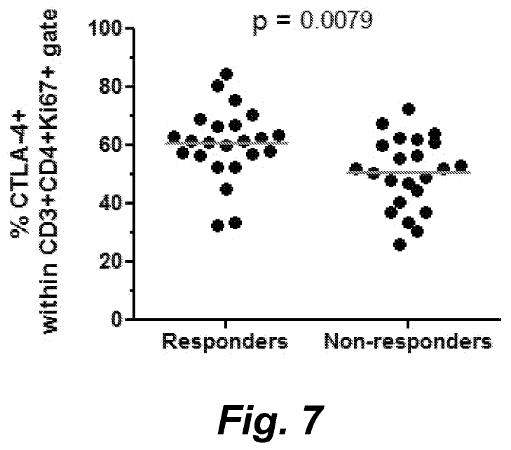

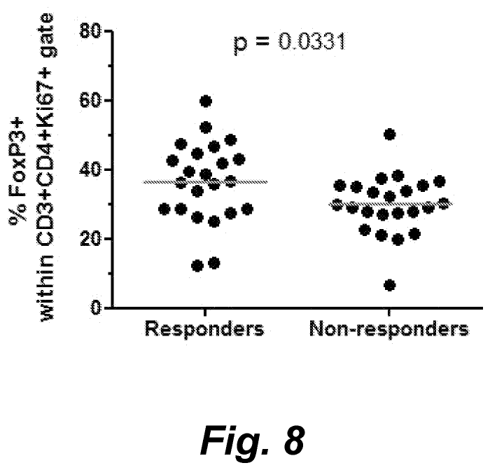

As shown in FIGS. 1-8, several distinct T cell subsets showed altered frequencies in clinical responders compared to non-responders. Specifically:
Proliferating (Ki67+) CD8+ T cells were more frequent in the circulation of patients who failed to respond to anti-PD1 therapy (FIG. 1)
The frequency of proliferating CD8+ T cells expressing the putative tumour-homing marker CD15s (also known as sialyl Lewis X, SSEA) was significantly higher in the circulation of anti-PD1 responders compared to non-responders (FIGS. 2-5). This trend was the same regardless of which anti-CD15s mAb clone was used to detect CD15s expression, although the percentage of cells staining positive varied for each clone. Note that the anti-CD15s clone ‘HECA-452’ recognises a number of CD15s variants collectively known as CLA (cutaneous lymphocyte antigen).
A similar trend was noted in the population of CD3+ T cells lacking expression of either CD4 or CD8 (double negative T cells). This population is presumed to consist primarily of γδ T cells. Within the proliferating (Ki67+) subset of this population, patients who responded to anti-PD1 had more CD15s+ cells, as detected using the HECA-452 mAb.
The frequency of proliferating CD4+ T cells expressing the inhibitory molecule CTLA-4 or the regulatory T cell marker FoxP3 was significantly higher for patients who responded to therapy (FIGS. 7-8). Both markers were detected intracellularly.

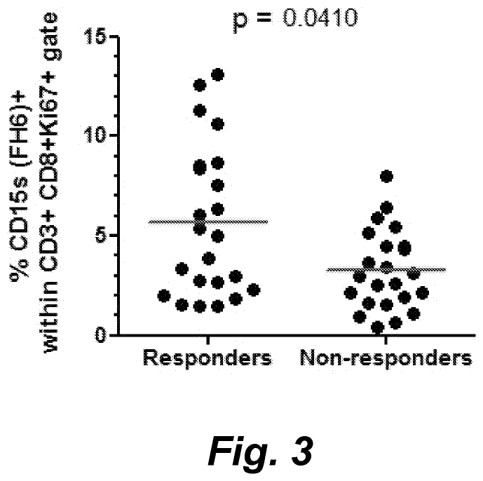

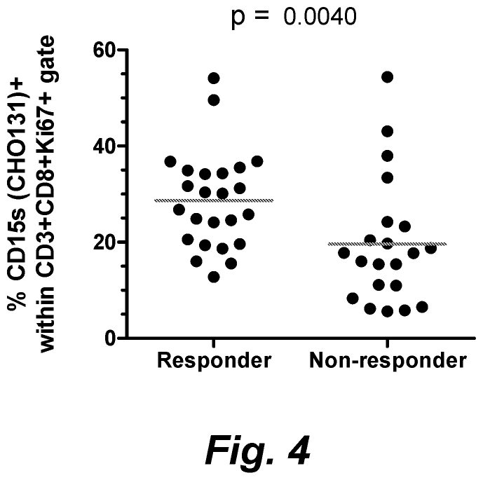

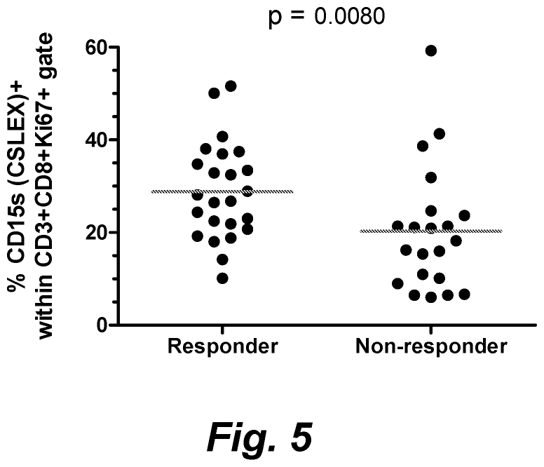

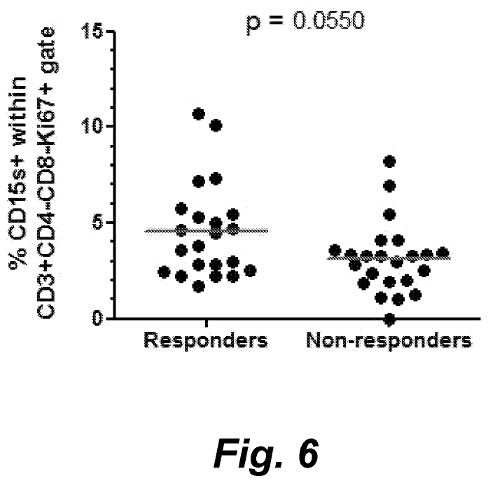

TABLE 2
Mean percentage of cells expressing markers

| | | Non- |
|---|---|---|
| | Responder | responder |
| FIG. 1 | 1.5% | 2.8% |
| (% CD3+/CD8+ cells expressing Ki67+) | | |
| FIG. 2 - HECA-452 clone | 19.2% | 10.3% |
| (% CD3+/CD8+/Ki67+ cells expressing CD15s) | | |
| FIG. 3 - FH6 clone | 5.3% | 3.5% |
| (% CD3+/CD8+/Ki67+ cells expressing CD15s) | | |
| FIG. 4 - CHO131 clone | 28.6% | 19.6% |
| (% CD3+/CD8+/Ki67+ cells expressing CD15s) | | |
| FIG. 5 - CSLEX clone | 28.8% | 20.2% |
| (% CD3+/CD8+/Ki67% cells expressing CD15s) | | |
| FIG. 6 - HECA-452 | 4.4% | 3.2% |
| (% CD3+/CD4−/CD8−/Ki67+ cells expressing | | |
| CD15s) | | |
| FIG. 7 | 60.4% | 50.1% |
| (% CD3+/CD4+/Ki67+ cells expressing CTLA-4) | | |
| FIG. 8 | 36.9% | 29.4% |
| (% CD3+/CD4+/Ki67+ cells expressing FoxP3) | | |

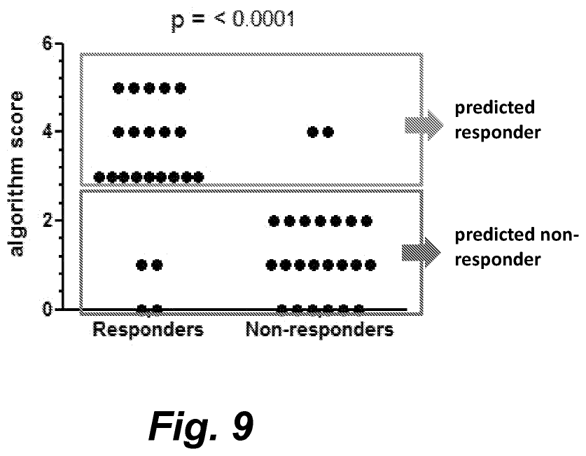

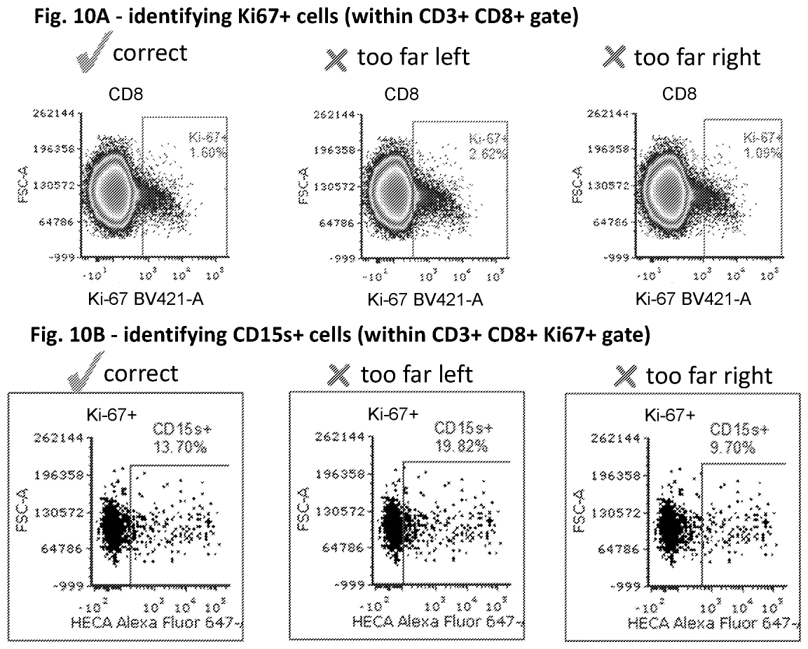

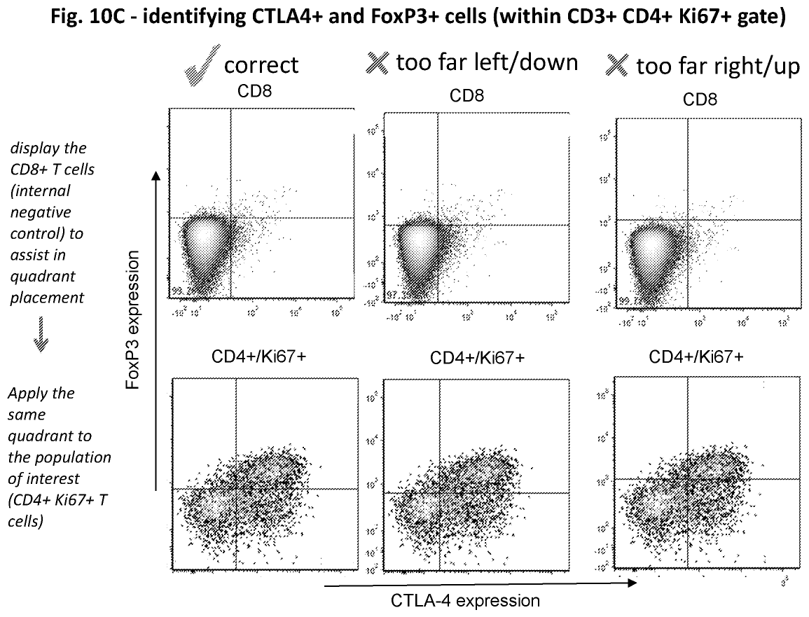

The results for several of these ‘signatures’ were combined into an algorithm designed to predict whether an individual patient would respond or not to anti-PD1 immunotherapy (FIG. 9). The algorithm was based on a points system for each of five signatures, whereby a point was allocated for each signature if a defined threshold value was reached. A patient scored 1 point for each of the following criteria:

$$> 16 \% CD 15 s + within CD 3 + C D 8 + Ki 67 + population ( using HECA ‐ 452 mAb to detect CD 15 s )$$

$$> 4 \% CD 15 s + within CD 3 + C D 8 + Ki 67 + population ( using FH 6 mAb to detect CD 15 s )$$

$$> 54 \% CTLA ‐ 4 + within CD 3 + CD 4 + Ki 67 + population$$

$$> 36 \% FoxP 3 + within CD 3 + CD 4 + Ki 67 + population$$

$$< 1.8 \% Ki 67 + within CD 3 + CD 8 + population .$$

The tally of points (score) for each patient is shown in FIG. 9. The difference in scores between responders and non-responders was highly significant by the Mann-Whitney test (p<0.0001). Patients scoring 3 or above were considered as predicted responders, while patients scoring 2 or below were considered as predicted non-responders.

The majority of responders (19/23) had scores of 3 or higher, whereas only 2/23 non-responders had scores of 3 or higher. It is anticipated that this approach could be used to accurately predict response to anti-PD1 therapy, with an overall accuracy of 87% in the current cohort (n=46).

### Conclusions

ICI drugs are changing the outlook for thousands of patients every year, providing the prospect of long term remissions or even cures for cancers that only a few years ago were highly lethal. A critical issue, though, is that these approaches work well for some patients and not at all for others. There is only limited knowledge of what makes some patients responsive and others not, and until now, no way to accurately predict into which category a particular patient will fall. Also lacking are effective biomarkers of treatment responsiveness that can be used to determine when treatment can be stopped for patients who are responding. The T cell signatures identified here appear to provide highly accurate prediction of response to anti-PD1 therapy prior to initiating treatment, and may also enable monitoring of responsiveness to therapy during the course of treatment. We also anticipate that this approach will perform similarly to predict responsiveness to anti-PDL1 drugs such as atezolizumab, because they target the same molecular pathway as the anti-PD1 drugs.

### Example 2—Analyses in Dissociated Metastatic Melanoma Tissue

We have also conducted analyses on three samples of dissociated metastatic melanoma tissues (from ICI-naïve patients). Proliferating CD8+ T cells expressing CD15s were detectable in all samples, and were strikingly enriched in one, with 57.5%, 5.3% or 2.8% of the CD8+CD28+Ki67+ T cells expressing CD15s (as detected using the FH6 mAb).

### Example 3—Determination of T Cell Subsets within Tumour Tissue

In one approach, high-content confocal microscopy may be used to detect T cells with a specific signature phenotype within sections of formalin-fixed paraffin embedded (FFPE) tumour tissue. For example, tissue sections may be analysed using 6-plex immunohistochemistry (IHC) using Opal™ chemistry and a Vectra imaging platform. Tissues will be stained for CD3, CD8, Ki67 and CD15s to identify the T cells of interest, together with melanoma-specific markers (eg SOX10, tyrosinase, gp100). Co-staining for CD31 and PDL1 will determine whether these T cells co-localise with vessels and/or PDL1+ cells, which may indicate clinically significant immune interactions. Image analysis (InForm software) will allow for tissue segmentation and cell phenotyping for quantitative and spatial analysis of cell subsets. This analysis may be used to assess CD15s+ T cells in tumour tissue, and it is anticipated that their frequency in tumour will relate to their frequency in blood and to response to ICI therapy.

### Example 4—Kits and Products for Assessing ICI Responsiveness

Kits and products for assessing ICI responsiveness may, for example, include one or more of the following components:
Blood collection tubes with sodium heparin or EDTA
Phosphate buffered saline (PBS)
A density gradient medium
Staining buffer (PBS+0.04% sodium azide+1% bovine serum albumin)
Fixation and permeabilisation reagents
Immunological reagents: for example primary and secondary antibodies may include one or more of the following:

TABLE 3
Antibodies useful in a kit for assessing ICI responsiveness

| Specificity | Conjugate | Clone | Supplier |
|---|---|---|---|
| CD3 | BUV395 | SK7 | BD ™ |
| CD4 | BV510 | SK3 | BD ™ |
| CD8 | BV786 | SK1 | BD ™ |
| Ki67 | BV421 | B56 | BD ™ |
| CD15s (dimeric) | PE | FH6 | BioLegend ™ |
| CD15s (conventional) | BV510 | CSLEX | BD ™ |
| CD15s (conventional | AF647 | HECA-452 | BD ™ |
| or sulphated) | | | |
| CD15s (conventional, | AF488 | CHO131 | R&D Systems ™ |
| linked to core 2 O- | | | |
| glycan) | | | |
| CTLA4 | PECy7 | 14D3 | Life |
| | | | Technologies ™ |
| FoxP3 | PerCP-Cy5.5 | PCH101 | Life |
| | | | Technologies ™ |

### Example 5—Protocols for Analysing Patient Samples

An exemplary protocol for analyzing samples for CD15s and CTLA-4 expression within a pathology lab is provided below

### Whole Blood Lysis and Fc Receptor Blocking

Four mls of blood was diluted in 46 ml of fresh 1× BD Pharm Lyse™, and mixed, followed by incubation at room temperate (RT) in accordance with the manufacturer's instructions. The sample was then centrifuge and the supernatant was removed before being resuspended in wash buffer. Following centrifugation, the supernatant was removed, and wash buffer was added up to a final volume of 200 μl. Fc receptors were blocked with human IgG (Thermo Fisher #02-7102) followed by incubation in accordance with the manufacturer's instructions.

### Surface Staining

Ninety μl (˜2×10⁶-3×10⁶ cells) of lysed blood sample was added to each tube to be stained for a marker (CD15s and CTLA-4) before mixing and incubation at RT in the dark. Stained samples were washed with 2 ml wash buffer followed by centrifugation and decanting of the supernatant. The pellet was then vortexed to loosen the cell pellets.

### Fixation and Permeabilisation (˜40 Min)

Fix/Perm solution was added to each tube followed by mixing and incubation at RT in the dark. Two ml of freshly prepared Permeabilization Buffer was added and then the samples were centrifuge and the supernatant decanted

### Blocking and Intracellular Staining (˜50 Min)

Four μl of normal mouse serum (Thermo Fisher #01-6501) was added to each tube followed by incubation at RT. Intracellular antibody cocktail mix was added to each tube and the samples were mixed and incubate at RT in dark. Two ml of Permeabilization Buffer was added before the cells were centrifuged and the supernatant decanted. Two ml of 1× Permeabilization Buffer was added, and the samples were centrifuged before the supernatant was decanted.

### Final Resuspension and Analysis

Each sample was resuspend in wash buffer and run on a flow cytometer

### Reagents

### CD15s SURFACE Antibody Mix

| | |
|---|---|
| BSB Plus | BD #566385 |
| CD45-APC-H7 | BD #560274 |
| CD3-BV510 | BD #563109 |
| CD8-PE-Cy7 | BD #557746 |
| CD4-PerCP-Cy5.5 | BD #560650 |
| CHO131-AF488 | R&D Systems #FAB996G |
| HECA-452-PE | BD #563962 |
| FH6-AF64 | Biolegend #368103 |

### CTLA-4 SURFACE Antibody Mix

| | |
|---|---|
| BSB Plus | BD #566385 |
| CD45-APC-H7 | BD #560274 |
| CD3-AF488 | BD #557694 |
| CD4-BV510 | BD #562970 |

### CD15s INTRACELLULAR Antibody Mix

| | |
|---|---|
| BSB Plus | BD #566385 |
| Ki67-BV421 | BD #562899 |

### CTLA-4 INTRACELLULAR Antibody Mix

| | |
|---|---|
| BSB Plus | BD #566385 |
| Ki67-BV421 | BD #562899 |
| CTLA-4-AF647 | Biolegend #349920 |
| FoxP3-PE | BD #560046 |

1× Fix/Perm solution (from FoxP3 buffer set: Thermo Fisher #00-5523-00)
1× Permeabilization buffer (from FoxP3 buffer set: Thermo Fisher #00-5523-00)

### Other Reagents Required:

Wash buffer (PBS+0.5% BSA+0.04% sodium azide)
BD Pharm Lyse (diluted according to manufacturer's recommendations, BD #555899)
Purified human Ig: Thermo Fisher #02-7102
Normal mouse serum Thermo Fisher #01-6501

### Example 6—Protocols for Analysing Patient Samples

Ki67 is an intracellular indicator of cell proliferation, being an indicator of the active phase of the cell cycle. However, due to its intracellular nature, to measure the level of Ki67 with in a cell, the cell must first be made permeable to allow intracellular access. Permeabilising cells, which typically also requires fixing of cell proteins, structures and antigens, adds additional time and cost to cell analysis. Therefore, it would be advantageous to identify a cell surface marker which can be substituted for Ki67.

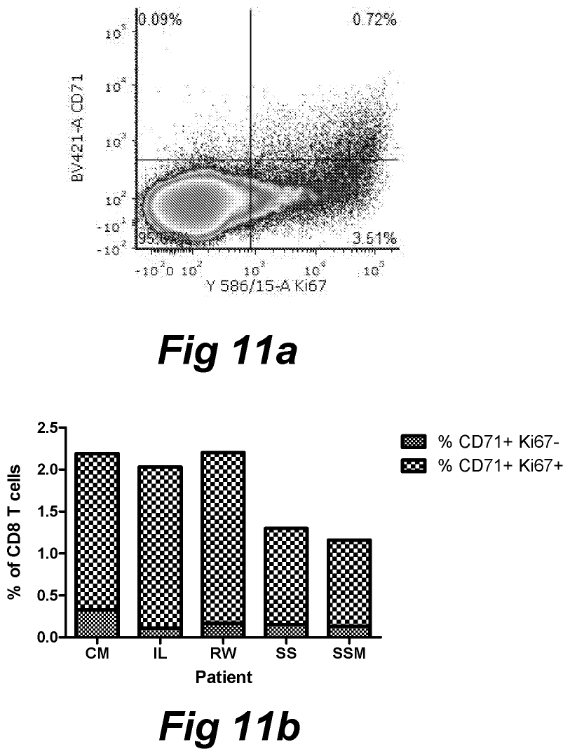

FIGS. 11a and 11b illustrate co-staining of cells for CD71 and Ki67.

Peripheral blood mononuclear cells were isolated from pre-treatment blood samples and subjected to immunostaining and flow cytometry analysis as described above. Following exclusion of dead cells (FVS575V+), CD8+ T cells were gated for Ki67 (horizontal axis of FIG. 11a) and CD71 (vertical axis of FIG. 11a). Within this gate, the percentage of cells staining positive for Ki67 and CD71 was determined.

FIG. 11b provides a representative plot from 5 melanoma patients (denoted CM, IL, RW, SS and SSM—i.e., patients 1 to 5), showing the percentage of CD71+CD8+ T cells that co-express Ki67. This demonstrates that a high percentage of cells expressing the CD71 marker are also Ki67+. However, as indicated in FIG. 11a, only a subset of Ki67+ cells co-express CD71. Therefore, it is proposed CD71 is a marker for a specific subset of Ki67 positive cells, that may have a significantly higher portion of the relevant markers such as CD15s, CTLA-4 and FoxP3.

### Definitions and Qualifications

As used through the specification and in the claims, the term “anti-PD1 immune checkpoint inhibitor immunotherapy” relates to any therapy which disrupts the signalling and/or activation pathway of Programmed Cell Death Protein 1 (PD1), also known as cluster of differentiation (CD) 279. This includes inhibitors of the PD1 protein (such a pembrolizumab and nivolumab) as well as inhibitors of its ligands (such as PD-L1—inhibited by avelumab and atezolizumab—or PD-L2). It is to be understood that the term encompasses both current and future inhibitors of PD1, its ligands, its signalling pathways and/or its activation pathways.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming to or through the present application, by claiming a divisional status and/or by claiming a continuation status from the present application. It is to be understood that the following claims are not intended to limit the scope of what may be claimed in any such future application. Nor should the claims be considered to limit the understanding of (or exclude other understandings of) the present disclosure. Features may be added to or omitted from the claims at a later date both within this application and within future applications.

The claims defining the invention are as follows:

1. A method of determining responsiveness of a subject suffering from, or susceptible to, a cancer to anti-PD1 immune checkpoint inhibitor immunotherapy, the method comprising assessing the vascular and/or tumour associated level in the subject of one or more of CD15s marker in Ki67+CD8+ T cells or CD71+CD8+ T cells, CTLA-4 marker in Ki67+CD4+ T cells or CD71+CD4+ T cells, FoxP3 marker in Ki67+CD4+ T cells or CD71+CD4+ T cells, CD15s marker in Ki67+CD4− CD8− T cells or CD71+CD4− CD8− T cells, and Ki67 marker in CD8+ T cells or CD71 marker in CD8+ T cells, wherein an increased level of the CD15s marker in the Ki67+CD8+ T cells or CD71+CD8+ T cells prior to the immunotherapy, an increased level of the CTLA-4 marker in said T cells prior to and/or after the immunotherapy, an increased level of the FoxP3 marker in said T cells prior to immunotherapy, an increased level of the CD15s marker in Ki67+CD4− CD8− T cells or CD71+CD4− CD8− T cells prior to immunotherapy, and a decreased level of the Ki67+ marker or CD71+ in said T cells prior to immunotherapy, is indicative that the subject is responsive to the immunotherapy.

2. The method according to any one of claim 1, wherein the Ki67+CD8+ T cells comprise Ki67+CD8+CD3+ T cells.

3. The method according to claim 1 or 2, wherein the Ki67+CD8+ T cells comprise Ki67+CD8+CD3+CD28+ T cells

4. The method according to any one of claims 1 to 3, wherein the Ki67+CD4+ T cells comprise Ki67+CD4+CD8− T cells.

5. The method according to any one of claims 1 to 4, wherein the Ki67+CD4+ T cells comprise Ki67+CD4+CD3+ T cells.

6. The method according to any one of claims 1 to 5, wherein the method comprises determining the level of one or more of the markers expressed by cells by flow cytometry.

7. The method according to any one of claims 1 to 6, wherein the method comprises use of one or more of FH6, HECA-452, CHO131, and CSLEX monoclonal antibodies to determine the level of the CD15s marker.

8. The method according to claim 7, wherein a level of the CD15s marker in Ki67+CD8+ T cells of 3.5% or greater determined using the FH6 monoclonal antibody (mAb) in a sample from the subject prior to immunotherapy is indicative that the subject is responsive to the immunotherapy.

9. The method according to claim 7 or 8, wherein a level of the CD15s marker in Ki67+CD8+ T cells of 10% or greater determined using the HECA-452 mAb in a sample from the subject prior to immunotherapy is indicative that the subject is responsive to the immunotherapy.

10. The method according to any one of claims 7 to 9, wherein a level of the CD15s marker in Ki67+CD8+ T cells of 18% or greater determined using the CHO131 mAb in a sample from the subject prior to immunotherapy is indicative that the subject is responsive to the immunotherapy.

11. The method according to any one of claims 7 to 10, wherein a level of the CD15s marker in Ki67+CD8+ T cells of 18% or greater determined using the CSLEX mAb in a sample from the subject prior to immunotherapy is indicative that the subject is responsive to the immunotherapy.

12. The method according to any one of claims 1 to 11, wherein a level of the CTLA-4 marker in Ki67+CD4+ T cells of 42% or greater in a sample from the subject prior to and/or after the immunotherapy is indicative that the subject is responsive to the immunotherapy.

13. The method according to any one of claim 1 to 12, wherein a level of the FoxP3 marker in Ki67+CD4+ T cells of 30% or greater in a sample from the subject prior to immunotherapy is indicative that the subject is responsive to the immunotherapy.

14. The method according to any one of claims 1 to 13, wherein a level of the CD15s marker in Ki67+CD4− CD8− T cells of 3% or greater in a sample from the subject prior to immunotherapy is indicative that the subject is responsive to the immunotherapy.

15. The method according to any one of claims 1 to 14, wherein a level of the Ki67+ marker in CD8+ T cells of 4% or less in a sample from the subject prior to immunotherapy is indicative that the subject is responsive to the immunotherapy.

16. The method according to any one of claims 1 to 15, wherein the level of the marker is compared to the level of the markers in a non-responsive subject, and/or a predetermined level of the marker.

17. The method according to any one of claims 1 to 16, wherein the method comprises determining the level of transcription of one or more of the markers.

18. The method according to any one of claims 1 to 17, wherein the method comprises determining the level of the one or more markers in the blood of a subject.

19. The method according to any one of claims 1 to 18, wherein the method comprises determining the level of the one or more markers in a tumour of a subject.

20. The method according to any one of claims 1 to 19, wherein the method comprises determining the level of the one or more markers of tumour infiltrating lymphocytes.

21. The method according to any one of claims 1 to 20, wherein the method further comprises use of a genetic characteristic of the cancer, and/or one or more genetic and/or clinical characteristics of the subject, to assess responsiveness of the subject to the immunotherapy.

22. The method according to any one of claims 1 to 21, wherein the method is used to select a subject suitable for immune checkpoint inhibitor mono-immunotherapy and/or to select a subject suitable for a non-immune checkpoint inhibitory immunotherapy.

23. The method according to any one of claims 1 to 22, wherein the cancer is selected from one of melanoma, non-small cell lung cancer, mesothelioma, kidney cancer, head and neck cancer, Hodgkin Lymphoma, Merkel cell carcinoma, bladder cancer, triple negative breast cancer, oesophageal cancer, gastric cancer, squamous cell carcinoma of the skin, cervical cancer, a cancer with microsatellite instability and/or mismatch repair enzyme deficiency, hepatocellular carcinoma, and primary mediastinal large B-cell lymphoma, and other malignancies susceptible to therapeutic immune checkpoint blockade.

24. The method according to any one of claims 1 to 23, wherein the method comprises using computer software executable by a processor to process data representative of the vascular and/or tumour associated level of one or more of the markers, and thereby provide a measure of the responsiveness of the subject to the immunotherapy.

25. A method of determining responsiveness of a subject suffering from, or susceptible to, a cancer to anti-PD1 immune checkpoint inhibitor immunotherapy, the method comprising assessing the level in the blood in the subject of one or more of CD15s marker in Ki67+CD8+ T cells or CD71+CD8+ T cells, CTLA-4 marker in Ki67+CD4+ T cells or CD71+CD4+ T cells, FoxP3 marker in Ki67+CD4+ T cells or CD71+CD4+ T cells, CD15s marker in Ki67+CD4− CD8− T cells or CD71+CD4− CD8− T cells, and Ki67 marker or CD71 marker in CD8+ T cells, wherein an increased level of the CD15s marker in the Ki67+CD8+ T cells or CD71+CD8+ T cells prior to the immunotherapy, an increased level of the CTLA-4 marker in said T cells prior to and/or after the immunotherapy, an increased level of the FoxP3 marker in said T cells prior to immunotherapy, an increased level of the CD15s marker in Ki67+CD4− CD8− T cells or CD71+CD4− CD8− T cells prior to immunotherapy, and a decreased level of the Ki67+ marker or CD71 marker in said T cells prior to immunotherapy, is indicative that the subject is responsive to the immunotherapy.

26. A method of determining responsiveness of a subject suffering from, or susceptible to, a cancer to anti-PD1 immune checkpoint inhibitor immunotherapy, the method comprising assessing the level in cells in a tumour in the subject of one or more of CD15s marker in Ki67+CD8+ T cells or CD71+CD8+ T cells, CTLA-4 marker in Ki67+CD4+ T cells or CD71+CD4+ T cells, FoxP3 marker in Ki67+CD4+ T cells or CD71+CD4+ T cells, CD15s marker in Ki67+CD4− CD8− T cells or CD71+CD4− CD8− T cells, and Ki67 marker or CD71 marker in CD8+ T cells, wherein an increased level of the CD15s marker in the Ki67+CD8+ T cells or CD71+CD8+ T cells prior to the immunotherapy, an increased level of the CTLA-4 marker in said T cells prior to and/or after the immunotherapy, an increased level of the FoxP3 marker in said T cells prior to immunotherapy, an increased level of the CD15s marker in Ki67+CD4− CD8− T cells or CD71+CD4− CD8− T cells prior to immunotherapy, and a decreased level of the Ki67+ marker or CD71 marker in said T cells prior to immunotherapy, is indicative that the subject is responsive to the immunotherapy.

27. A method of determining responsiveness of a subject suffering from, or susceptible to, a cancer to anti-PD1 immune checkpoint inhibitor immunotherapy, the method comprising assessing the level in tumour infiltrating lymphocytes in the subject of one or more of CD15s marker in Ki67+CD8+ T cells or CD71+CD8+ T cells, CTLA-4 marker in Ki67+CD4+ T cells or CD71+CD4+ T cells, FoxP3 marker in Ki67+CD4+ T cells or CD71+CD4+ T cells, CD15s marker in Ki67+CD4− CD8− T cells or CD71+CD4− CD8− T cells, and Ki67 marker or CD71 marker in CD8+ T cells, wherein an increased level of the CD15s marker in the Ki67+CD8+ T cells or CD71+CD8+ T cells prior to the immunotherapy, an increased level of the CTLA-4 marker in said T cells prior to and/or after the immunotherapy, an increased level of the FoxP3 marker in said T cells prior to immunotherapy, an increased level of the CD15s marker in Ki67+CD4− CD8− T cells or CD71+CD4− CD8− T cells prior to immunotherapy, and a decreased level of the Ki67+ marker or CD71 marker in said T cells prior to immunotherapy, is indicative that the subject is responsive to the immunotherapy.

28. A method of treating a subject suffering from, or susceptible to, a cancer with an anti-PD1 immune checkpoint inhibitor immunotherapy, the method comprising identifying a subject responsive to the immune checkpoint inhibitor immunotherapy using the method according to any one of claims 1 to 27, and treating the subject so identified with the immune checkpoint inhibitor.

29. A method of treating a subject suffering from, or susceptible to, a cancer with an anti-PD1 immune checkpoint inhibitor immunotherapy, the method comprising:
determining responsiveness of the subject to the immune checkpoint inhibitor immunotherapy on the basis of the vascular and/or tumour associated level of one or more of CD15s marker in Ki67+CD8+ T cells or CD71+CD8+ T cells, CTLA-4 marker in Ki67+CD4+ T cells or CD71+CD4+ T cells, FoxP3 marker in Ki67+CD4+ T cells or CD71+CD4+ T cells, CD15s marker in Ki67+CD4− CD8− T cells or CD71+CD4− CD8− T cells, and Ki67 marker or CD71 marker in CD8+ T cells, wherein an increased level of the CD15s marker in the Ki67+CD8+ T cells or CD71+CD8+ T cells prior to the immunotherapy, an increased level of the CTLA-4 marker in said T cells prior to and/or after the immunotherapy, an increased level of the FoxP3 marker in said T cells prior to immunotherapy, an increased level of the CD15s marker in Ki67+CD4− CD8− T cells or CD71+CD4− CD8− T cells prior to immunotherapy, and a decreased level of the Ki67+ marker or CD71 marker in said T cells prior to immunotherapy, is indicative that the subject is responsive to the immunotherapy; and
treating the subject determined to be responsive with the immunotherapy.

30. A kit for use in assessing responsiveness of a subject suffering from, or susceptible to, a cancer to anti-PD1 immune checkpoint inhibitor immunotherapy; the kit comprising one or more of an immunological reagent for detecting CD15s marker, an immunological reagent for detecting CTLA-4 marker, an immunological reagent for detecting FoxP3 marker, and one or both of an immunological reagent for detecting Ki67 and an immunological reagent for detecting CD71.

31. Use of one or more of CD15s, CTLA-4, FoxP3, Ki67 and CD71 as a marker to assess the responsiveness of a subject suffering from, or susceptible to, a cancer to anti-PD1 immune checkpoint inhibitor immunotherapy.

32. A method of assessing responsiveness of a subject suffering from, or susceptible to, a cancer to anti-PD1 immune checkpoint inhibitor immunotherapy, the method comprising:
receiving data representative of the vascular and/or tumour associated level of one or more of CD15s marker in Ki67+CD8+ T cells or CD71+CD8+ T cells, CTLA-4 marker in Ki67+CD4+ T cells or CD71+CD4+ T cells, FoxP3 marker in Ki67+CD4+ T cells or CD71+CD4+ T cells, CD15s marker in Ki67+CD4− CD8− T cells or CD71+CD4− CD8− T cells, Ki67 in CD8+ T cells and CD71 in CD8+ T cells; and
processing the data to determine the responsiveness of the subject to the immunotherapy.

33. Software for use with a computer comprising a processor and memory for storing the software, the software comprising a series of instructions executable by the processor to carry out the method according to claim 32.

34. A system for assessing responsiveness of a subject suffering from, or susceptible to, a cancer to anti-PD1 immune checkpoint inhibitor immunotherapy, the system comprising
a flow cytometer;
a processor;
a memory; and
software resident in the memory and accessible to the processor, the software comprising a series of instructions executable by the processor to carry out the method according to claim 32.

35. A system for assessing responsiveness of a subject suffering from, or susceptible to, a cancer to anti-PD1 immune checkpoint inhibitor immunotherapy, the system comprising
a means for determining the level of one or more of CD15s marker in Ki67+CD8+ T cells or CD71+CD8+ T cells, CTLA-4 marker in Ki67+CD4+ T cells or CD71+CD4+ T cells, FoxP3 marker in Ki67+CD4+ T cells or CD71+CD4+ T cells, CD15s marker in Ki67+CD4− CD8− T cells or CD71+CD4− CD8− T cells, Ki67 in CD8+ T cells and CD71 in CD8+ T cells, in a blood and/or tumour associated sample from a subject;
a processor;
a memory; and
software resident in the memory accessible to the processor, the software comprising a series of instructions executable by the processor to process data representative of the vascular and/or tumour associated level of the one or more of the markers, and thereby provide a measure of the responsiveness of the subject to the immunotherapy.

36. A system for assessing responsiveness of a subject suffering from, or susceptible to, a cancer to anti-PD1 immune checkpoint inhibitor immunotherapy, the system comprising
a flow cytometer;
a processor;
a memory; and
software resident in the memory accessible to the processor, the software comprising a series of instructions executable by the processor to process data received from the flow cytometer representative of the vascular and/or tumour associated level of the CD15s marker in Ki67+CD8+ T cells or CD71+CD8+ T cells, CTLA-4 marker in Ki67+CD4+ T cells or CD71+CD4+ T cells, FoxP3 marker in Ki67+CD4+ T cells or CD71+CD4+ T cells, CD15s marker in Ki67+CD4− CD8− T cells or CD71+CD4− CD8− T cells, Ki67 in CD8+ T cells and CD71 in CD8+ T cells, and thereby provide a measure of the responsiveness of the subject to the immunotherapy.
